(19) United States
(12) Patent Application Publication
(10) Pub. No.: US 2024/0285345 A1
(43) Pub. Date: Aug. 29, 2024

(54) INTEGRATED SURGICAL THEATER REPRESENTATION, PLANNING, AND COORDINATION

(71) Applicant: Intuitive Surgical Operations, Inc., Sunnyvale, CA (US)

(72) Inventors: Gaurav Lamba, Fremont, CA (US); Kenneth Lee, Oakton, VA (US); Angus Jonathan McLeod, Sunnyvale, CA (US); Sundar Murugappan, Danville, CA (US); Govinda Payyavula, Sunnyvale, CA (US); Pourya Shirazian, Menlo Park, CA (US)

(21) Appl. No.: 18/587,498

(22) Filed: Feb. 26, 2024

Related U.S. Application Data
(60) Provisional application No. 63/449,010, filed on Feb. 28, 2023.

(51) Int. Cl. A61B 34/10; A61B 34/37; A61B 90/00; G06F 30/20

(52) U.S. Cl. CPC A61B 34/10; A61B 34/37; A61B 90/37; G06F 30/20; A61B 2034/104; A61B 2034/105; A61B 2034/107; A61B 2090/365

(57) ABSTRACT

Various of the disclosed embodiments provide systems and methods for coordinating actions among team members within a surgical theater, including robotic surgical theaters. For example, embodiments may create a three-dimensional model of the patient's interior, or a portion of the patient's interior, using data from an imaging device coupled to a surgical instrument. This model may then be used to facilitate more coordinated decisions among team members in the theater, such as the placement of additional laparoscopic ports. Augmented reality and virtual reality planning systems and methods availing themselves of the three-dimensional model, including “virtual dollhouse” roleplaying methods, may also facilitate team member coordination in some embodiments. Models of the surgical environment may also provide predictive analyses so as to extend the surgical team's “planning horizon.” Such predictive analytics may itself benefit from historical surgical data, including past records of patient interior model creation and planning.

### CROSS-REFERENCE TO RELATED APPLICATIONS

This application claims the benefit of and priority to U.S. Provisional Patent Application No. 63/449,010, filed Feb. 28, 2023, entitled “INTEGRATED SURGICAL THEATER REPRESENTATION, PLANNING, AND COORDINATION”, which is incorporated by reference herein in its entirety for all purposes.

### TECHNICAL FIELD

Various of the disclosed embodiments relate to systems and methods for representing patient interiors and for coordinating surgical planning so as to avoid undesirable surgical theater configurations and surgical outcomes.

### BACKGROUND

Even experienced surgeons and surgical staff can have difficulty coordinating their time-sensitive actions within the complex environment of a surgical theater. Simply communicating the current state of the surgery to one another may be difficult, as team members may assume different positions and roles throughout the surgical procedure, precipitating an asymmetric distribution of information about the surgical state among the team. For example, in a robotically assisted surgery, surgical operators of the robot may have a superior visualization of a surgical site as compared to the team members responsible for swapping instruments upon the robot. However, the team members managing the instrument swaps may themselves have a superior understanding of the surgical robot's orientation within the theater as a whole as compared to the robot's operator. Thus, the operator may better appreciate constraints occurring within the surgical site, whereas the other team members may better appreciate constraints occurring between equipment and personnel in the theater as a whole. Verbally communicating these disparate understandings between the team members in a timely manner may be difficult, frustrating, and ineffective.

Difficulties resulting from such poor communication may themselves initiate a cascade of undesirable consequences. For example, team members may disagree about where to place a laparoscopic port based upon their subjective understandings of the surgical situation. If an improper placement occurs, e.g., denying the operator adequate instrument reach or visibility of a desired anatomy, the team may then be required to remove the instrument from the original port, create an additional port, perform unplanned repositioning of the surgical robot's arms, as well as undock and redock the surgical robot. These time-consuming actions may themselves then precipitate further difficulties, e.g.: the surgery may now be undesirably prolonged, subjecting the patient to additional anesthesia than expected, as well as increase the team members' fatigue; each port removal and insertion exposes the patient to the risk of a port site infection; downstream surgical tasks anticipating the originally planned configuration may now need to be adjusted, possibly with unforeseen consequences; significant delay may require rescheduling of downstream operations planned for the same theater; etc. Poor contextual communication not only increases the likelihood of such undesirable situations, but may likewise complicate their resolution and the team's recovery. Indeed, lack of a common context may often constrain the team's predictive horizon, making it difficult for team members to appreciate the consequences of their respective choices upon one another, upon the future state of the patient, and upon the future state of the theater.

Many potential solutions fail to satisfactorily address these difficulties. For example, robot manufacturers have sought to avoid some of these outcomes by educating team members regarding the robotic system's operational parameters and limitations, going so far as to provide printed guides and handouts for the team's reference during the procedure. These guides may seek to anticipate problematic instrument and arm selections, helping the team members to proactively avoid undesirable combinations of arm positions, port placements, and instruments. Unfortunately, team member turnover, the many types of undesirable configurations, the large and continually changing corpus of surgical instruments, the particular character of any given patient or theater, etc., mitigate the pedagogical effectiveness of these approaches. Instead, many personnel often either apply a “one size fits all” port placement approach, neglecting the specifics of a given situation and patient anatomy, or the personnel “guesstimate” the linearity and distance for a particular port placement, estimating the instrument reach with only a hazy, or no, recollection of the manufacturer's previously provided guidelines. As instrument lengths may vary considerably, such ad hoc approaches are rarely consistent. General rules of thumb likewise often result in inconsistent outcomes when performing the “same” surgery, given the variability of patient anatomies, instrument lengths, theater configurations, etc.

One might also hope that optimization algorithms, constraint based selection methods, applications in transport theory, etc. would suffice to resolve in-theater coordination. Unfortunately, translating these analyses from the rarefied atmosphere of theory into the real-world context of the theater can be difficult, particularly when seeking actionable information in a dynamic context for personnel with a wide variety of backgrounds and experiences. Such methods may also be restrictive in terms of the procedure or criteria considered for their optimization functions, failing to anticipate for the dynamic character of the real-world environment. With respect to port placement specifically, such algorithms may provide only the optimized port placement configuration on the interior wall of the patient anatomy, without providing additional contextual information to help the team address unexpected theater constraints (e.g., the presence of other equipment in the theater, the dimensions of the theater, an ad hoc configuration of the robot arms, etc. may all be unanticipated by the optimization algorithm). Indeed, the patient's anatomy is sometimes dynamic and unexpected movement or relocation of a target anatomy may precipitate the need for port relocation unanticipated by the optimization algorithm. Even when such optimization methods identify efficient approaches, it may be difficult for personnel to visualize the surgical field so as to execute the identified solution.

Accordingly, there exists a need for systems and methods to overcome challenges and difficulties such as those described above. For example, there exists a need for systems and methods able to assist surgical teams and reviewers to consistently orient and navigate within patient interiors as well as within the wider surgical theater context.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation, as may occur in relation to some embodiments;

FIG. 1B is a schematic view of various elements appearing in a surgical theater during a surgical operation employing a surgical robot, as may occur in relation to some embodiments;

FIG. 2A is a schematic representation of a series of surgical procedures and surgical tasks during a surgical procedure, as may occur in some embodiments;

FIG. 2B is a schematic flow diagram providing a general overview of various steps in a surgical theater coordination process, as may be implemented in various embodiments;

FIG. 3A is a schematic representation of a surgical robot with a single, initial instrument inserted into the patient, as may occur in some embodiments;

FIG. 3B is a schematic representation of a single imaging device, such as a colonoscope, as may be used in some embodiments;

FIG. 3C is a schematic representation of a stereoscopic imaging device, as may be used in some embodiments;

FIG. 3D is a pair of images depicting a checkered board in perspective view from a planar imaging device field of view and from a fish-eye imaging device field of view, respectively, as may occur in some embodiments;

FIG. 3E is a schematic representation of a patient interior including relative positions of a target anatomy and an imaging device, as may occur in some embodiments;

FIG. 3F is a schematic representation of a panning operation within the patient interior of FIG. 3E for the purposes of preparing an interior model of at least a portion of the patient interior, as may occur in some embodiments;

FIG. 3G is a pair of schematic interior model cross sections, one model without in-filling and the other model with in-filling, as may occur in some embodiments;

FIG. 4 is a flow diagram illustrating various operations in an example mapping process, as may be implemented in some embodiments;

FIG. 5A is a schematic view of virtual elements internal and external to an interior model, as well as a corresponding surgical robot configuration prior to a port placement proposal, as may be implemented in some embodiments;

FIG. 5B is a schematic view of virtual elements internal and external to an interior model, as well as a corresponding surgical robot configuration, during a first port placement proposal for a first robotic arm in the procedure of FIG. 5A, as may be implemented in some embodiments;

FIG. 5C is a schematic view of virtual elements internal and external to an interior model, as well as a corresponding surgical robot configuration, during a second port placement proposal for a second robotic arm in the procedure of FIG. 5A, as may be implemented in some embodiments;

FIG. 6A is a schematic view of an outer range indication virtual element representation, as may be implemented some embodiments;

FIG. 6B is a patient interior model with example port placement virtual element recommendation representations, as may be implemented in some embodiments;

FIG. 6C is a schematic view of an example collection of instrument degrees of freedom as may inform the conical representations in, e.g., FIGS. 6D and 6E, in some embodiments;

FIG. 6D is a schematic view of a conical instrument range representation virtual element, as may be implemented in some embodiments;

FIG. 6E is a schematic view of a composite conical instrument range representation virtual element, as may be implemented in some embodiments;

FIG. 6F is a schematic view of a patient interior model with example port placement recommendation representation virtual elements, as well as corresponding conical instrument range representation virtual elements, as may be implemented in some embodiments;

FIG. 7A is a flow diagram illustrating various operations in an example process for rendering an external robotic arm range representation virtual element, as may be implemented in some embodiments;

FIG. 7B is a flow diagram illustrating various operations in an example process for rendering an internal instrument range representation virtual element, as may be implemented in some embodiments;

FIG. 8A is a schematic representation of a target anatomy with a corresponding spherical virtual element, as may be implemented in some embodiments;

FIG. 8B is a schematic representation of a target anatomy and a corresponding composite virtual element, as may be implemented in some embodiments;

FIG. 8C is a schematic representation of two intersecting virtual conic volumes corresponding to ranges of motion for two instruments relative to their respective proposed portal entries, as may be implemented in some embodiments;

FIG. 8D is a schematic representation of the target anatomy and corresponding composite virtual element of FIG. 8B, in connection with a volumetric intersection of a virtual conical instrument range volume element, as may be implemented in some embodiments;

FIG. 8E is a schematic representation of the target anatomy and corresponding composite virtual element of FIG. 8B, in connection with a surface intersection of an conical instrument range volume virtual element, as may be implemented in some embodiments;

FIG. 8F is a flow diagram illustrating various operations in an example intersection rendering process, as may be implemented in some embodiments;

FIG. 9 is a flow diagram illustrating various operations in an example process for registering historical data, such as computed tomography (CT), magnetic resonance imaging (MRI), ultrasound, etc., data previously acquired for the patient, as may be implemented in some embodiments;

FIG. 10A is a flow diagram illustrating various operations in an example process for determining specific port placement recommendations, as may be implemented in some embodiments;

FIG. 10B is a flow diagram illustrating various operations in an example process for determining general port placement recommendations, as may be implemented in some embodiments;

FIG. 11A is a schematic representation of a surgical robotic system with personnel employing various augmented reality (AR) systems, as may be implemented in some embodiments;

FIG. 11B is a schematic representation of the field of view within an operator console for a surgical robot in the theater of FIG. 11A, as may be implemented in some embodiments;

FIG. 11C is a sequence of images showing the state of an augmented reality representation for exterior personnel within the theater of FIG. 11A, from a first perspective, as may be implemented in some embodiments;

FIG. 11D is a sequence of images showing the state of an augmented reality representation for exterior personnel within the theater of FIG. 11A, from a second perspective, as may be implemented in some embodiments;

FIG. 12 is a schematic collection of graphical user interface elements as may appear in a surgical composition and planning management application, as may be implemented in various embodiments;

FIG. 13A is a schematic block diagram illustrating various relations between various virtual groupings within the surgical theater, as may be considered in various embodiments;

FIG. 13B is a schematic block diagram illustrating various relations between elements in “task-role-action” and “task-role-AR virtual element” tables, as may be implemented in some embodiments;

FIG. 13C is a schematic block diagram illustrating various data structures in an example global representation data structure of the surgical theater, as may be implemented in some embodiments;

FIG. 13D is a flow diagram illustrating various operations in an example process for managing role-based virtual element renderings and interaction within a surgical theater, as may be implemented in some embodiments;

FIG. 14A is a schematic representation of team member viewing a virtual representation of a patient interior from a first perspective, and consequent rendering adjustments, as may be implemented in some embodiments;

FIG. 14B is a schematic representation of the team member of FIG. 14A viewing the virtual representation of the patient interior from a second perspective, and consequent rendering adjustments, as may be implemented in some embodiments;

FIG. 14C is a flow diagram illustrating various operations in an example role-aware virtual element rendering process, as may be implemented in some embodiments;

FIG. 15A is a flow diagram illustrating various operations in a first example augmented reality registration process, as may be implemented in some embodiments;

FIG. 15B is a flow diagram illustrating various operations in a second example augmented reality registration process, as may be implemented in some embodiments;

FIG. 15C is a flow diagram illustrating various operations in a third example augmented reality registration process, as may be implemented in some embodiments;

FIG. 16A is a schematic representation of a virtual camera view as may be presented in an operator console of a surgical robotic system, or upon a display in the theater for multiple team members to review, in some embodiments;

FIG. 16B is a flow diagram illustrating various operations in an example process for managing a virtual camera rendering, as may be implemented in some embodiments;

FIG. 17 is a flow diagram illustrating various operations in an example process for operation-wide computer-assisted theater planning, as may be implemented in some embodiments;

FIG. 18 is a flow diagram illustrating various operations in an example process for history-based virtual theater creation, as may be implemented in some embodiments;

FIG. 19A is a schematic block diagram of various components in a surgery assessment framework, as may be used in some embodiments;

FIG. 19B is a flow diagram illustrating various operations in an example surgery assessment process, as may be implemented in some embodiments;

FIG. 20 is a flow diagram illustrating various operations in an example visual record search process, as may be implemented in some embodiments; and

FIG. 21 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

### Example Surgical Theaters Overview

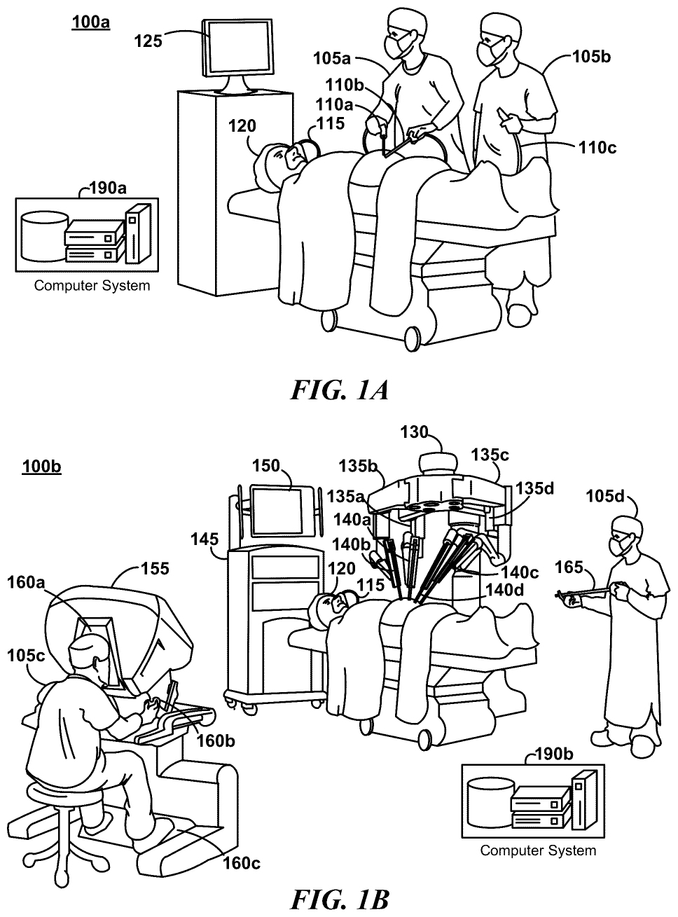

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image acquiring endoscope, etc. and a mechanical instrument 110a such as scissors, retractors, a dissector, etc.

The visualization tool 110b provides the surgeon 105a with an interior view of the patient 120, e.g., by displaying visualization output from an imaging device mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is an endoscope, one will appreciate that, in some embodiments, visualization tool 110b may capture depth data instead of, or in addition to, two-dimensional image data (e.g., with a laser rangefinder, stereoscopy, etc.). Accordingly, one will appreciate that it may be possible to apply various of the two-dimensional operations discussed herein, mutatis mutandis, to such three-dimensional depth data when such data is available.

A single surgery may include the performance of several groups of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a surgical robot, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc. the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement within display 160a, etc. may all be recorded. Similarly, patient side cart 130 may record tool activations (e.g., the application of radiative energy, closing of scissors, etc.), movement of instruments, etc. throughout the surgery. In some embodiments, the data may have been recorded using an in-theater recording device, which may capture and store sensor data locally or at a networked location (e.g., software, firmware, or hardware configured to record surgeon kinematics data, console kinematics data, instrument kinematics data, system events data, patient state data, etc., during the surgery).

Within each of theaters 100a, 100b, or in network communication with the theaters from an external location, may be computer systems 190a and 190b respectively (in some embodiments, computer system 190b may be integrated with the robotic surgical system, rather than serving as a standalone workstation). As will be discussed in greater detail herein, the computer systems 190a and 190b may facilitate, e.g., planning of the surgical procedure, data consolidation, and communication between the team members.

### Example Surgical Theater Task Workflow Context Overview

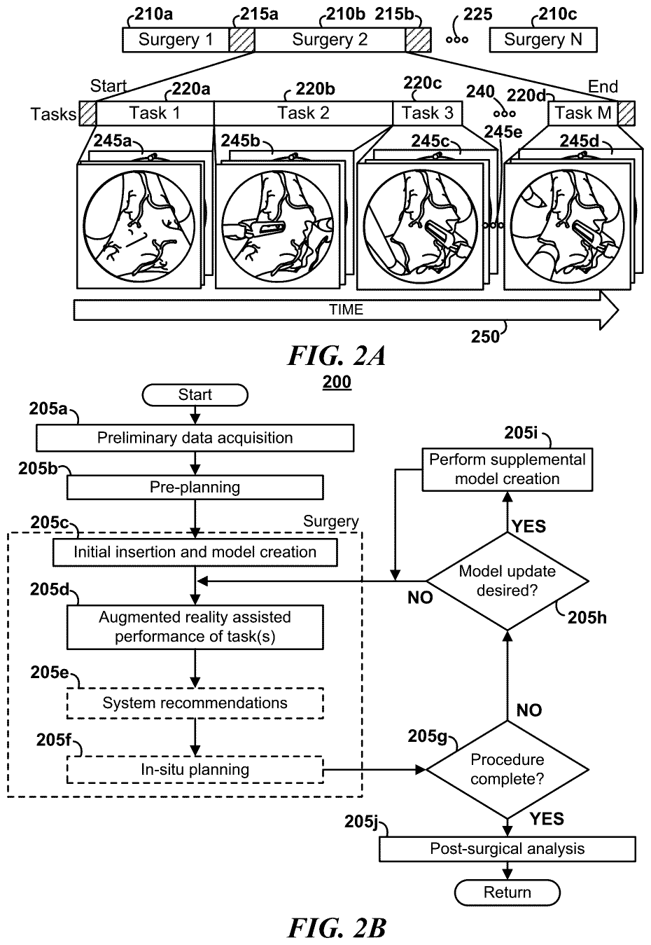

For the reader's context, FIG. 2A schematically depicts a variety of tasks occurring during one of a plurality of surgeries scheduled within a surgical theater. Specifically, the theater may be scheduled for surgeries 210a, 210b, and 210c, over time 250 (as well as intervening surgeries represented by ellipsis 225). Each of surgeries 210a, 210b, and 210c may include a plurality of tasks. For example, surgical procedure 210b includes the tasks 220a, 220b, 220c, and 220d (additional intervening tasks may be present, as indicated by ellipsis 240). Task 220a may be the “insert first imaging device system” task involving the placement of the first port and insertion of a laparoscopic imaging device so that the surgeon may acquire an initial view of the surgical site within the patient interior. Image frames (e.g., read-green-blue “RGB” images) captured with one or more such imaging devices (e.g., RGB cameras) may be organized into sets associated with each task. For example, the set 245a is here generated during task 220a, the set 245b is generated during task 220b, the set 245c is generated during task 220c, and the set 245d is generated during task 220d. Kinematic data from the surgeon console, such as the motion of the control inputs by the surgeon, the motion of the surgeon's eyes, pedal motions by the surgeon, etc. may also be captured in connection with each of sets 245a-d (with intervening sets represented by ellipsis 245e). Sets 245a-d may also include kinematics data for the robotics system, such as arm motion, instrument motion, instrument activation, instrument arm swaps, etc. System event data, such as replacement of an instrument, activation of a tool (such as a cauterizer), etc., may also be collected in connection with the data sets 245a-d.

One will appreciate that task start and end times may be chosen so as to allow temporal overlap between tasks, or may be chosen to avoid such temporal overlaps. For example, in some embodiments, tasks may be “paused” as when a surgeon engaged in a first task transitions to a second task before completing the first task, completes the second task, then returns to, and completes, the first task. Accordingly, while start and end points may define task boundaries, one will appreciate that data may be annotated to reflect timestamps affiliated with more than one task.

Between each surgery there may be a preparation period, e.g., periods 215a, 215b, during which personnel change over, patients leave and enter, surgical tools are sterilized and collected for the next surgery, etc. Thus, one will appreciate, e.g., that delays or adverse events (e.g., multiple port placement attempts) in surgery 210a may delay the beginning of, and increase the turnaround time of, period 215a, possibly resulting in delay and potential rescheduling of the remaining surgeries, e.g., surgeries 210b and 210c.

### Overview of Example Surgical Theater Integration Methodology

Various embodiments provide planning in the context of a collaborative visual exploration and design space to avoid various adverse events in the surgical theater, such as those discussed above. Enabling surgeons and staff to visually analyze, collaborate, and plan surgical actions, both before and during a surgery, facilitates more robust surgical strategy planning, decreases the likelihood of adverse events, and facilitates recovery from the adverse events when they occur. Some such systems and methods may anticipate the disparate and dynamic characters of surgical theaters, specifically eschewing procrustean algorithmic solutions which fail to properly account for team member feedback during the surgery. For example, various of the disclosed approaches invite personnel participation so as to overcome the reciprocal “chicken and egg” constraints of internal instrument range of motion and external robotic arm range of motion in a specific surgical context. Personnel may “roleplay” the surgery with virtual elements before the surgery and then perform their respective roles within the agreed context during the surgery (e.g., eschewing programmatic solutions in favor of a “virtual dollhouse” discussion using virtual elements as described in greater detail herein). In this manner, e.g., the team may organically modify an initially imperfect surgical plan through their discussion, thereby overcoming the reciprocal “chicken and egg” constraints (e.g., the plan may initially satisfy neither set of constraints, the team may then roleplay and discuss using the virtual or augmented reality “dollhouse” until the first set of constraints are satisfied, and then the team may explore further incremental adjustments until all the constraints are satisfied).

For clarity, one will appreciate that reference herein to “virtual” models and elements, or to “representations” or “virtual representations” of various items, refers not only to elements which may be rendered in a virtual reality environment (e.g., wherein the elements are not rendered so as to appear within a real-world imaging device capture), but to elements which may be rendered in an augmented reality environment (e.g., wherein the virtual element is, e.g., scaled or synthetically occluded so as to appear to the viewer as if the element resided within a real-world imaging device capture). For example, one will appreciate that a model in a Wavefront™ OBJ 3D geometry file format may be rendered in both virtual and augmented reality environments. Similarly, one will appreciate that there may be many equivalent ways to render a virtual element or representation within a rendering pipeline. For example, where a virtual element or representation is to be depicted as an outline around a portion of a surface of a mesh, the virtual element or representation may be a change in the texture rendering upon that portion of the mesh, a vertex extrusion around that portion of the mesh, a texture rendering on a two-dimensional plane (e.g., a billboard surface) positioned in the three-dimensional rendering pipeline field of view so as to appear to the viewer as an outline upon the mesh, a second mesh of a differentiating texture placed upon the first mesh, etc. Additionally, one will appreciate that as virtual elements and representations appear in data structures (e.g., the Wavefront™ OBJ file format, JavaScript Object Notation (JSON) format, Extensible Markup Language (XML), etc.) a computer system may receive, analyze, or manipulate a representation of an item or a virtual element without necessarily rendering the representation or the virtual element.

FIG. 2B is a schematic flow diagram providing a general overview of various steps in a surgical theater coordination process 200, as may be implemented in various embodiments. While specific reference is often made herein to the example of port placement so as to facilitate the reader's understanding, one will appreciate that various of the embodiments presented herein may facilitate coordination of other aspects of the surgical theater (e.g., the ordering of surgical tasks, cauterization and excision orderings, the placement of fiducials, etc.). Here, process 200 reflects operations various embodiments may employ to facilitate coordination among team members, not unlike musicians in a symphony performing their individual functions while remaining aware of the wider thematic context.

As will be described in greater detail herein, elements of process 200 may facilitate improved coordination, e.g., for tasks, such as port placement, by coordinating full visualization information regarding the surgical site across members of the surgical team. These operations may facilitate port placement strategies sufficiently robust so as to accommodate diverse patient populations and procedure types (herniotomy, colectomy, prostatectomy, etc.), while also accommodating optimization to a specific patient and theater's circumstances. Embodiments may also facilitate port placement selections in shorter periods of time than previously achieved, as well as better facilitate consistent outcomes and port choices when confronted with similar surgical conditions.

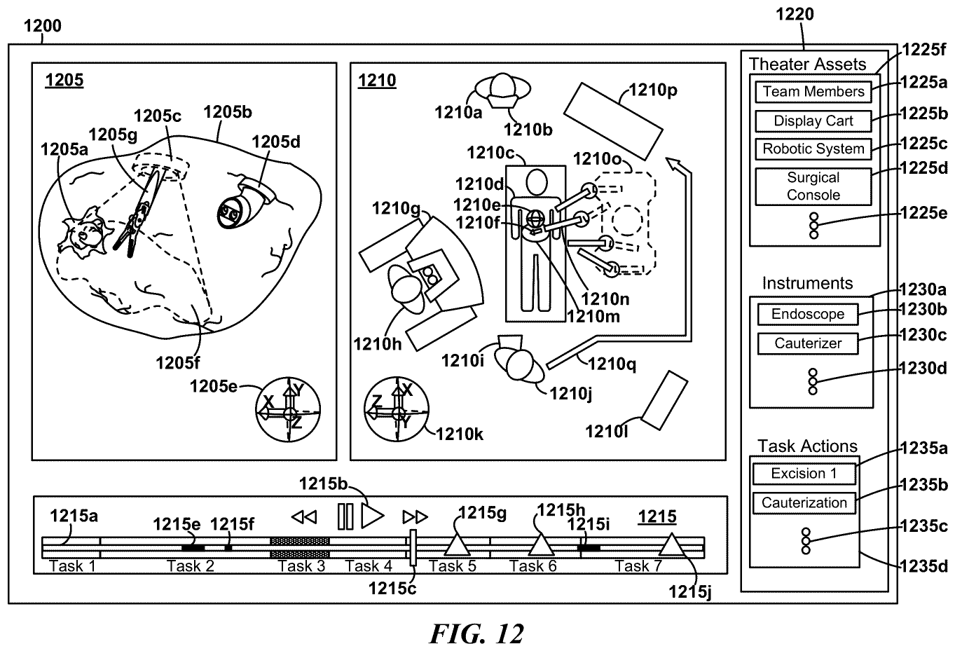

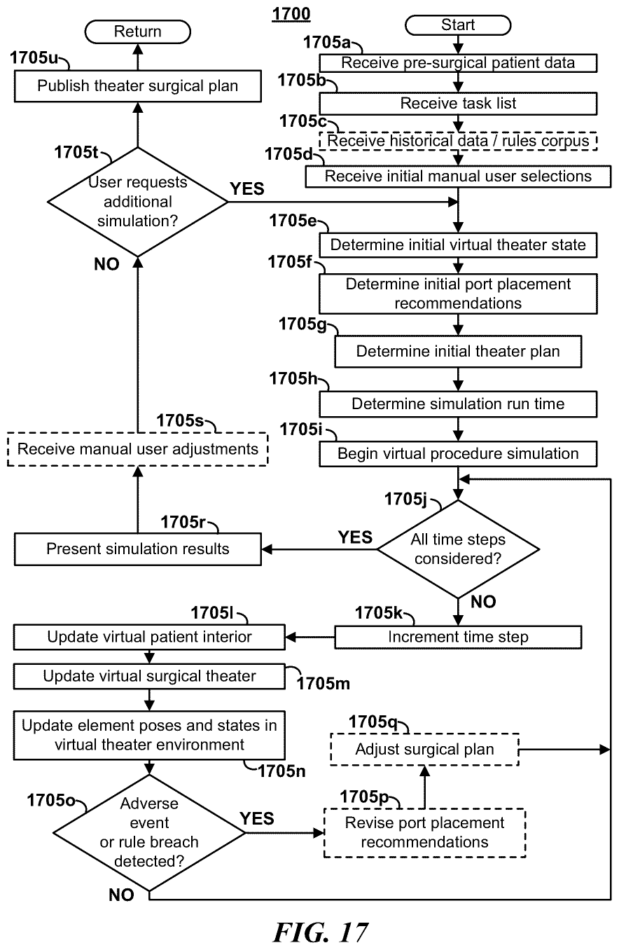

Initially, at block 205a, a computer system (e.g., one of systems 190a, 190b, or an out-of-theater desktop, laptop, or network system, or a combination of the two) may perform preliminary data acquisition. For example, the computer system may acquire computed tomography (CT) scans of the patient about to undergo surgery from a medical database, patient health history data, an inventory of the equipment and personnel that will be available in the theater at the scheduled time, etc. During pre-planning, at block 205b, one or more team members may coordinate member and equipment actions as will be described in greater detail here with respect to FIGS. 12 and 17. Such coordination of theater configurations may be memorialized in a record, referred to herein as a surgical plan (implemented, e.g., as a JSON file, XML file, etc., listing, e.g., a task ordering, various corresponding properties, task-role-AR and task-role-action tables, etc., as described herein). As will be described in greater detail, surgical planning may occur in one session or in multiple session iterations, the latter resulting in revision of the surgical plan both before and during the surgery. For example, team members may initially plan an original workflow for their procedure in surgery 210b. However, unexpected delays during the previous procedure 210a may require a timeline or inventory adjustment for surgery 210b. Accordingly, while surgery 210a is still ongoing, adjustments may be made to surgery 210b's surgical plan in a second planning session in anticipation of the new timeframe and adjusted theater environment. Similarly, if an adverse event occurs during surgery 210b (e.g., time delay resulting from a workstation collision with a robotic arm, complications occurring during a tumor excision, etc.), the surgical plan may be modified (e.g., preferred, but unnecessary, tasks at the surgery's conclusion may be removed from the plan) via in-situ planning (as will be discussed with respect to block 205f).

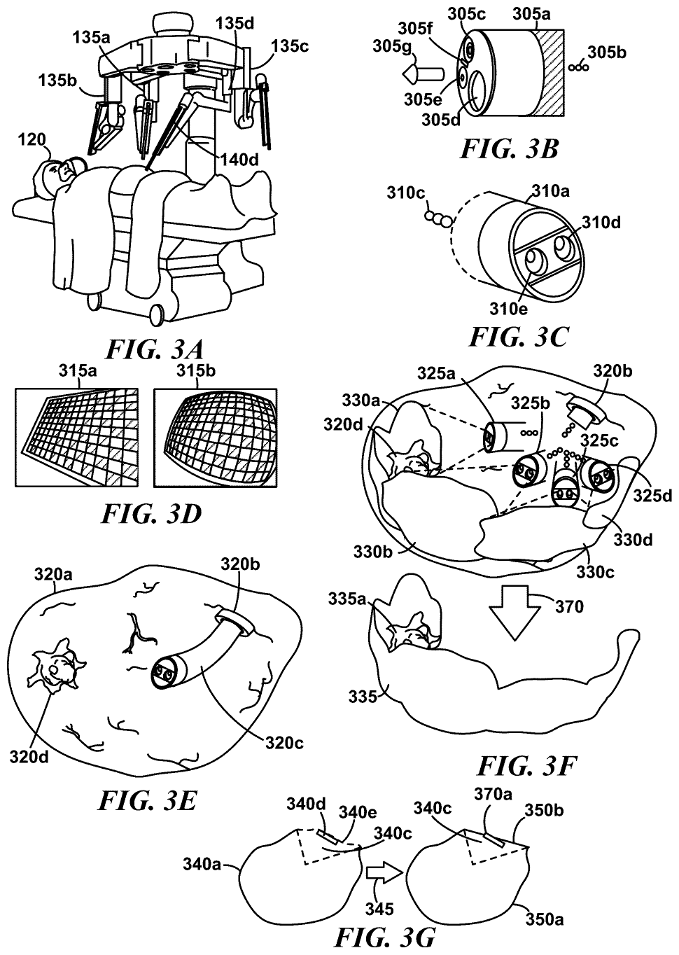

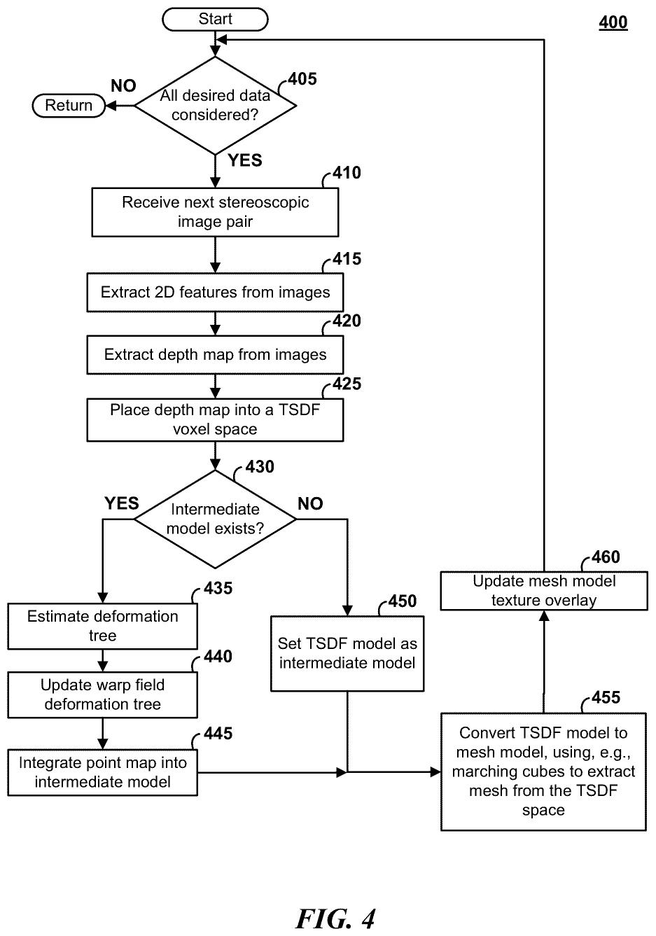

Once the surgery (e.g., surgery 210b) has begun, at block 205c, the team members may perform an initial imaging device insertion and patient interior model creation process, e.g., as will be described herein in greater detail herein with respect to FIGS. 3A-G and FIG. 4. This process may create a three-dimensional model of all, or a portion, of the patient's interior at the surgical site, referred to herein as an interior model, which may then be referenced for a variety of analytic, diagnostic, virtual, and augmented reality operations as will be described in greater detail herein (e.g., a predicted volume, or surface concavity, of the interior model may be correlated with an insufflation level). CT scans and other data acquired at blocks 205a and 205b may inform the initial imaging device insertion of block 205c (e.g., having generally identified the location of a target anatomy from a CT scan, the approximate location in the patient's body may inform the team's initial port placement).

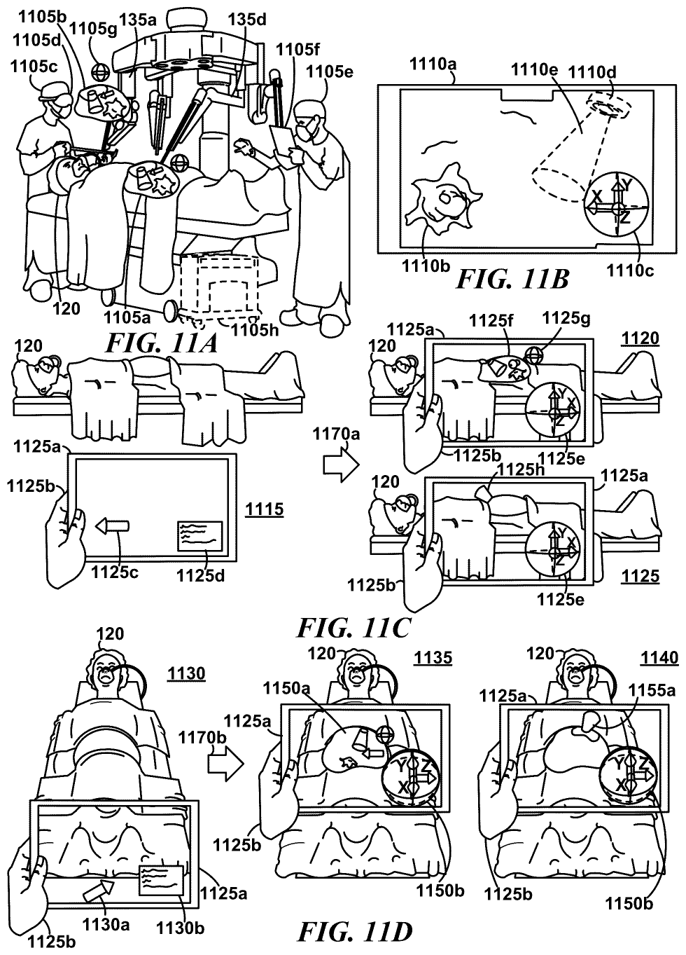

All or a portion of the interior model, or of augmented reality elements, may be rendered, e.g., upon display 125, display 150, or display 160a. Specifically, at block 205d, the interior model may be used in conjunction with a variety of augmented reality operations and elements, as will be described herein (e.g., with respect to FIGS. 11A-D), to facilitate coordinated interactions between the team members in accordance with the current version of the surgical plan developed at block 205b (or updated at block 205f).

In some embodiments, during the surgery, at block 205e, or as part of pre-planning block 205b, or in-situ planning at block 205f, a computer system may provide various recommendations to team members as new data is acquired or as the computer system or team members adjust the surgical plan. For example, as will be described in greater detail herein, with a complete, or at least sufficiently complete, model of the patient interior as it appears during the actual surgery, port placement recommendations may now be possible so as to assist the team in more efficiently continuing their surgical plan. Similarly, in some embodiments, at block 205f, the system or users may engage in additional planning during the surgery, e.g., when unexpected events occur. For example, if the surgery was initially only intended to biopsy a first tumor, but the scan at block 205c revealed a second, previously unknown, tumor requiring immediate removal, then at block 205f, the surgical plan may be modified to accommodate the additional tumor removal (e.g., with the insertion of additional corresponding tasks).

Per blocks 205b and 205f the system may take advantage of historical data (e.g., collected in past surgeries via devices such as the Intuitive Surgical, Inc. dvLogger™, etc.), insights generated from such data (e.g., surgical scene segmentation, surgical performance analytics, etc.) and human reasoning to provide a comprehensive visualization of the entire surgical space both inside and outside the patient body in conjunction with a set of interactive tools allowing the surgical team to interact, analyze, and explore this surgical space collaboratively. Such collaboration may facilitate initial port placement selections, additional access point selections for additional instruments, robot placements and configurations, poses and positioning movement for the surgical team members throughout the procedure, etc. For clarity, a “pose” refers to the translational position and rotational orientation of a body. For example, in a three-dimensional space, one may represent a pose with six total degrees of freedom. One will readily appreciate that poses may be represented using a variety of data structures, e.g., with matrices, with quaternions, with vectors, with combinations thereof, etc. Thus, in some representations, when there is no rotation, a pose may comprise only a translational component. Conversely, when there is no translation, a pose may comprise only a rotational component.

Where the surgical procedure has not yet completed at block 205g, the process 200 may transition to block 205h for consideration whether updates to the current model (e.g., as created at block 205c) would be suitable, or if new models are desired. For example, the surgical team may relocate the imaging device to a new location (or insert another imaging device) and request creation of a new interior model from the computer system. Similarly, while respiration and other patient motion may generate some minor deviation during model creation, detecting deviations beyond a threshold (e.g., when a particle filter consistently fails to align the existing model with newly acquired depth data) may precipitate a model update. Patient anatomy may also change within the surgical site over the course of the operation, e.g., as various tasks are performed (such as an excision), likewise inviting updated or refreshed models. In these situations, new or supplemental model operations may be performed at block 205i, replacing all or a portion of the interior model based upon more recently acquired data.

After the surgery has finished at block 205g, at block 205j, post-surgical analysis may be performed, e.g., to maintain a record of surgeries as part of the patient's medical record for future reference, to analyze surgeon and surgical team performance, for team members to create rules preventing adverse events that occurred, or nearly occurred, during his surgery from occurring in future operations (e.g., a particular combination of instruments and arm-instrument assignments should not be used to access anatomy within a particular region of the patient), etc. This post-surgical analysis and data storage may be informed, e.g., by the models generated at blocks 205c and 205i, the augmented reality elements generated at block 205d, recommendations and corresponding analysis at block 205e, as well as the surgical plans prepared at blocks 205b and 205f as described in greater detail herein. In combination with the kinematics and system events data often acquired in the theater, as with robotic surgical systems, this may create a rich corpus of data from which many useful inferences may be drawn (e.g., for team feedback, additional rule creation and rule qualification, etc.).

### Example Patient Interior Mapping

FIG. 3A is a schematic representation of a surgical robot with a single instrument inserted into the patient, as may occur in some embodiments, e.g., during the initial patient interior surgical mapping, as may occur at block 205c. Specifically, when surgery first begins, albeit, after insufflation, a team member may perform an initial sensor insertion so as to assess the state of the patient interior, including, e.g., to validate the team's assumptions and inferences about the state of the patient from blocks 205a and 205b. Thus, initially, the robotic arms 135a-c and their corresponding instruments, which are not associated with the examining instrument 140d, may not be positioned so as to interact with the patient 120. Accordingly, in the depicted example, the visualization tool 140d upon arm 135d is positioned for patient insertion, whereas the arms 135a, 135b, and 135d are positioned away from the patient. Note that the current positions of arms 135a, 135b, and 135d may not be arbitrarily oriented, but rather, oriented in accordance with the surgical plan determined at block 205b as will be described in greater detail herein (as the chosen configuration may avoid collisions with other equipment, better anticipate receipt of an upcoming instrument swap, etc.).

One will appreciate that visualization tool 140d may assume a variety of forms and configurations. For example, FIG. 3B is a schematic representation of a single imaging device instrument 305a, such as a colonoscope, while FIG. 3C is a schematic representation of a stereoscopic imaging device 310a (e.g., an Intuitive Surgical, Inc. 3DHD® Vision stereoscopic endoscope), as may be used in some embodiments. One will appreciate that such instruments may be actuated so as to orient their imaging devices in a variety of directions. Consequently, the ellipsis 305b and the ellipsis 310c each indicate that the body of the respective instrument continues behind the imaging device. As the instrument 305a has only a single imaging device sensor 305c, it may have space to accommodate an additional instrument bay 305d, irrigation outlet 305f, and light source 305e. In some embodiments, one will appreciate that bay 305d may include a depth sensor (such as a time-of-flight photonic-based depth sensor). In many embodiments, however, depth-based models of the patient interior may be created from visual images, such as from a temporal sequence or spatial pair of visual color or grayscale images.

Where the imaging device instrument is a monocular imaging device, as in instrument 305a (as may occur, e.g., in the Intuitive Surgical, Inc. Ion™ system), motion of the imaging device may be used to acquire pairs of images for performing a depth determination. As the robotic system may acquire kinematics data associated with the imaging device position during its movement, precise correspondences between the acquired images and the imaging device's pose may be available to facilitate localization. For example, by advancing the instrument 305a forward in the direction 305g or backwards in the opposite direction, multiple images may be captured of the patient interior with knowledge of the imaging device's relative orientation at the time of capture. This relative orientation may facilitate corresponding depth determinations by comparing features in the respective images captured in the respective poses.

Rather than rely on temporally successive image captures to infer depth, example instrument 310a employs a pair of stereoscopic imaging device sensors 310e and 310d (e.g., to facilitate a stereoscopic presentation at display 160a). Since the distance between each imaging device sensors 310e and 310d remains fixed, one may infer depth by comparing the images of the stereoscopic image pair (e.g., identifying feature correspondences between the images, assessing the parallax via optical flow, etc.). While instrument 310a, which employs a stereoscopic imaging device system, will be commonly referenced herein to facilitate understanding, one will appreciate that any instrument facilitating depth data acquisition may suffice for many of the embodiments disclosed herein.

For clarity, one will appreciate that processing operations may be performed upon the images before or after inferring depth. For example, FIG. 3D is a pair of images depicting a checkered board in perspective view from a planar (also referred to herein as a Cartesian) imaging device perspective (shown in image 315a) and a fish-eye imaging device perspective (shown in image 315b), respectively, as may occur in some embodiments. Imaging device coupled instruments, such as those similar to instruments 305a and 310a, may employ either of the fish-eye or the planar, Cartesian views. While fish-eye views may provide the operator with a wider viewing range, the Cartesian view may be less cognitively tasking. With appropriate transformations, one will appreciate that depth may be inferred from pairs of images, regardless of the particular view used (e.g., fish-eye view, the planar Cartesian view, etc.), when the poses of the relative image captures are known.

FIG. 3E is a schematic representation of a patient interior 320a including relative positions of a target anatomy 320d (e.g., a tumor, a region to be surgically operated upon, a region to be explored, etc.) and an imaging device 320c (e.g., instrument 310a), as may occur in some embodiments. Again, one will appreciate that depending upon the kind of procedure or task being performed, the initial port placement may be upon different portions of the patient's anatomy and the location of the target anatomy may vary. As shown, a team member may have first introduced a laparoscopy port 320b through which the imaging device 320c (e.g., corresponding to the distal tip of visualization tool 140d) may enter the patient interior 320a. Selection of the orientation and placement of laparoscopic port 320b may have been in anticipation of target anatomy 320d being located at the depicted location (e.g., based upon previously acquired CT scans of the patient at block 205a).

Once inserted into the patient interior 320a, the operator may begin panning, or rotating, etc., the instrument 320c's field of view to capture images of the patient interior 320a so as to form an interior model (e.g., in accordance with a Strategic Localization and Mapping “SLAM” methodology as discussed in greater detail herein) completely, or partially, representing the patient interior (though many of the disclosed embodiments will be discussed herein with respect to SLAM so as to facilitate the reader's comprehension, one will appreciate a variety of other suitable methods for preparing an interior model, e.g., working with a Structure from Motion “SfM” algorithm, working with actual time-of-flight depth data from a range finder, combinations of these approaches, etc.). In some embodiments, such panning may be explicitly performed and directed, whereas in some embodiments, the panning may occur naturally as the operator assesses the patient interior or performs a task (e.g., searching for a target anatomy). For clarity, FIG. 3F is a schematic representation of an example panning operation within the patient interior 320a of FIG. 3E for the purposes of preparing an interior model of at least a portion of the patient interior 320a. Here, the instrument has assumed four poses 325a-d within the patient interior (which, again, may be recorded in connection with the corresponding kinematics data). In this example, the instrument includes two imaging device sensors and so each of poses 325a-d will provide a pair of images suitable for depth determination. Specifically, the pose 325a results in a depth data acquisition 330a, the pose 325b results in a depth data acquisition 330b, the pose 325c results in a depth data acquisition 330c, the pose 325d results in a depth data acquisition 330d (though not shown, one will appreciate that intermediate data captures may occur between the poses shown; similarly, the imaging devices may be panned or rotated in planes other than the horizontal plane and around axes other than the “yaw” axis shown in this example). In some embodiments, arrows or other directional indicators may be overlaid upon the console operator's view directing the operator to pan the sensor(s) in directions where unmapped portions of the patient interior remain, or where regions of the interior model are not up to date (e.g., discrete surface areas of the model may be associated with their own timers so that “staleness” of the captured data may be monitored over time). While the interior model may comprise the integrative combination of such depth captures, as in this example, in some embodiments the interior model may be a “local, live” model, consisting of only a single depth capture (or an integration of only a threshold number of previous captures), such as the most recent depth capture from the instrument 320c, discarding older captures from the interior model. While perhaps less comprehensive than an integrated interior model, a local, live interior model may more readily inform team members via the augmented reality systems disclosed herein of the present state of the operator's inspection, as well as the state of the patient interior. Consequently, in some embodiments, the team may alternate between an integrated interior model augmented reality rendering and a local, live interior model rendering throughout the task or procedure (e.g., as one or the other rendering becomes more relevant to a given action or task). Naturally, one will appreciate that where data is acquired for an integrated model, the integrated model may be rendered as if it were a “local, live” model virtual element.

Note that in this example integrated interior model creation the target anatomy 320d was captured in the depth data acquisition 330a, and so its surface will comprise a portion of the depth data acquisition 330a. Also note, as will be discussed in greater detail herein with respect to the SLAM methodology, that the depth data acquisitions 330a-d encompass overlapping portions of the patient interior 320a. Consequently, when they are merged 370 into a single portion of a three-dimensional virtual interior model 335, a portion 335a of the surface of the model will correspond to the target anatomy 320d. While the merge 370 may be performed on all the data acquisitions 330a-d one will appreciate that the model may also be produced iteratively (e.g., first combining acquisitions 330a and 330b, combining that result with acquisition 330c, and combining that result with acquisition 330d). Once a sufficiently complete model is available, some embodiments may compute characteristics of the patient interior, e.g., the surgical field volume to verify the state of insufflation. Similarly, once the model of the patient interior has been created, or at least sufficiently updated, corresponding analyses and augmented reality renderings may be possible. For example, the volume, structure, and texture of the model may be used to inform the character of the patient's current state, and such information may then be broadcast to the augmented reality devices of other surgical team members as well as used to update the surgical plan (e.g., selecting one of several plans based upon the severity of the patient's condition).

Again, for clarity, one will appreciate that the imaging device and mapping process may not capture the entirety of the patient's interior region. For example, many instruments may not be able to rotate in such a fashion as to view the region around the entry portal 320b. FIG. 3G is a pair of schematic interior model cross-sections 340a, 350a, the model associated with cross-section 340a without in-filling and the model associated with cross-section 350a with in-filling, as may occur in some embodiments. Specifically, it may not be possible for an imaging device instrument to rotate so as to view the surface 340e around the entry portal 340d falling within region 340c. Thus, when completing or rendering 345 the model, the computer system may replace the unknown surface with a planar in-fill surface 350b (or, in some embodiments, leave the region transparent or empty, particularly where the size of region 340c is relatively small in comparison to the regions of interest, e.g., around target anatomy 320d; accordingly, in some embodiments, the interior model may more resemble a surface, rather than an enclosure, as in many of the depicted examples). Where a second depth-acquiring instrument is available (e.g., later in the procedure), it may be possible to combine the depth data from each imaging device to in-fill the lacuna, if desired. Similarly, in some embodiments, artificial artist renderings of the presumed hidden surface may be provided or generated (e.g., via a human artist or via a neural network trained to perform three-dimensional in-painting).

As will be discussed in greater detail herein, even where the details of region 340c are not known for certain, a representation 370a of the initial port placement may be useful for discerning the relative position of target anatomy and other potential port placements (again, whether in an integrated interior model or in a local, live interior model). This location may be identified in the region 340c, or upon plane 350b, based upon the known articulation and insertion state of the imaging device relative to the model captures.

### Example Patient Interior Mapping—Example Mesh Creation Process

FIG. 4 is a flow diagram illustrating various operations in an example patient interior mapping process 400 for preparing an integrated interior model, as may be implemented in some embodiments. While data remains to be considered at block 405, the system may acquire the next set of images at block 410 from which depth data is to be determined. In this example, the images are a pair of stereoscopic images (though, again, in some embodiments, successive images from a monocular imaging device in motion, time of flight depth data from a depth sensor, etc. may be acquired in lieu of the operations depicted here in blocks 410, 415, and 420).

For the stereoscopic image pair considered here, at block 415 two-dimensional features (e.g., Scale Invariant Feature Transform “SIFT”, Features from Accelerated Segment Test “FAST”, etc.) may be extracted from the each of the images and their relative correspondence used at block 420 to infer depth values. The system may then integrate this new depth data with the existing model (e.g., the iterative merging of depth data 330a-d into model 335), e.g., placing the depth map into a Truncated Signed Distance Function “TSDF” voxel space at block 425. One will appreciate that each depth map is “relative” to the pose of the instrument and, thus, the system may translate the data to the coordinate system of the TSDF.

As discussed, in some embodiments, voxels may be removed over time, or voxels within a threshold distance of one another may be considered and only those with a most recent timestamp retained (the temporal character of corresponding mesh vertices may likewise be considered). Here, at block 430 the system considers whether this is the first iteration of the mapping operation (or, if a temporal “freshness” constraint is in effect, if all the previous voxels have expired). If so, then there is no present “intermediate model” (e.g., this is the first depth data frame 330a considered prior to integration of depth data frames 330b-d) and at block 450 the system may initialize the TSDF representation of the model with the data produced at block 425.

Conversely, if there is preexisting TSDF data in the model (e.g., depth data frames 330a-c have been integrated and the system is now considering depth data frame 330d), then at block 435 the system may estimate a deformation tree for the model and new data. The computer system may register the incoming data frame to the intermediate model using both two-dimensional texture features as well as three-dimensional shape in accordance, e.g., with deformable SLAM. Where deformable SLAM is used, the system may generate a warping function (a “dense” three-dimensional vector field) matching the incoming frame to the intermediate model, at block 440, and then integrate the TSDF point map into the intermediate TSDF model at block 445 (for clarity, points sharing a voxel may be combined into a single point based upon an average distance).

In some embodiments, the model may be rendered volumetrically in the TSDF form. Where, however, at block 455 a corresponding vertex mesh to the TSDF model is created, a texture, determined from the color or grayscale images themselves from block 410, may be used to texture the model at block 460. Again, since kinematics data, e.g., from a robotic system, may identify the instrument's pose relative to the surgical theater, the model can be oriented in this global coordinate frame so as to correspond with its real-world location within the theater.

### Example Patient Interior Mapping—Example Interior and Exterior Correspondence

Once the interior model has reached a sufficient state of completeness (e.g., following in-filling so as to form a complete model), various diagnostics, analytic, an augmented reality operations may be performed. Indeed, some analytics and augmented reality representations may begin even as the model is being created. As will be described in greater detail herein, e.g., with reference to port placement planning and recommendations, various embodiments may coordinate multiple representations of the patient's interior to facilitate interaction among the various team members.

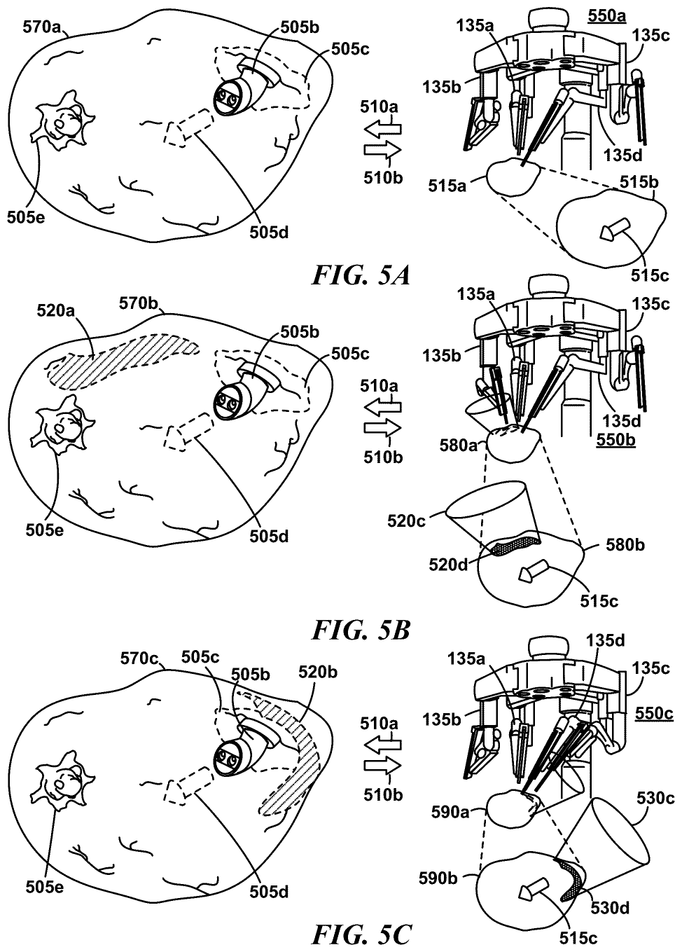

For example, FIG. 5A is a schematic view of a patient interior model 570a and a corresponding surgical robot configuration prior to a port placement proposal, as may occur in some embodiments. Here, as in FIG. 3A, only a single imaging device has been inserted via arm 135d in the robotic arm configuration 550a. As discussed, the target anatomy surface 505e may appear as part of the surface of the interior model 550a and the region 505c around the entry portal 505b may be in-filled or left empty. In some embodiments, the interior model 570a may include a virtual representation of the instrument 505b in its current position. Though shown here as a three-dimensional model of the instrument 505b, one will appreciate that simplified presentations, such as arrows, or geometric shapes, may also be used (e.g., the arrow 505d). As will be discussed, while team members may be able to view the interior model 570a from a variety of perspectives, the operator 105c of the surgeon console 155, or those viewing the imaging device's output on, e.g., display 125, may see the patient interior model (or only the virtual elements appearing therein or relative to the model) from the perspective of the imaging device's current pose (e.g., where the real-world imaging device image of target anatomy 505e has been highlighted by the operator or by the system, the highlight appearing as an overlay upon the imaging device output).

As will be discussed, complementary views of the interior model 570a and associated virtual elements may be provided to inform various team members of the relative states of various theater components. For example, various members may perceive an external model 515a, also shown for the reader's convenience in enlarged form 515b, based upon the interior model 570a. Rather than render the interior texture of the patient acquired via the imaging device as in the interior model 570a, exterior model 515b may render the exterior of the interior model (or may not itself be rendered, instead depicting virtual elements at locations upon or around the patient's exterior). Such an exterior representation may help team members to recognize the relationship between the patient interior and various elements of the surgical theater exterior to the patient. In some embodiments, the exterior model 515b, like interior model 570a, may include an arrow 515c or other suitable identifier to indicate the current pose of the instrument imaging device attached to arm 135d (again, in some embodiments, the virtual element for arrow 515c may be rendered without rendering the model). Again, in some embodiments, a corresponding rendering 505d may appear in the interior model, in combination or alternatively to the instrument rendering 505b.

In some embodiments, compasses may be provided within the rendering to help viewers orient the rendering relative to a global coordinate system in the theater (such as a coordinate system originating, e.g., from a fixed point upon the robotic system or a location in the theater). Thus, when the operator at the surgeon console comments upon the state of the surgery (e.g., “the target anatomy is to my right”), fellow team members may readily translate the comment to the global coordinate system (e.g., “the target anatomy is along the longitudinal axis of the patient, above the navel and below the rib cage”). Thus, observations, analysis, etc. made by the surgical team in connection with the exterior model 515b and exterior virtual elements may correspond 510a with the interior model 570a and internal virtual elements. Conversely, consideration of the interior model 570a and internal elements may correspond 510b with the external model 515b and external virtual elements. Similarly, these elements may correspond with elements presented in the display 160a (to the operator 105c) or display 150 (or display 125 in a non-robotic theater) in the perspective of the current imaging device pose.

Such correspondences may be particularly useful when considering future port placements. For example, as indicated in state configuration 550a, the instruments of robotic arms 135a, 135b, and 135c are presently unused. However, users may select one or more of the robotic arms as well as corresponding instruments, or the system may automatically select one or more for the arms, as will be discussed in greater detail herein, for addition port placement consideration. While such selections may be performed via a graphical interface, in some embodiments, as a team member moves a robot arm's instrument within a threshold distance of the patient interior, as is the case with robotic arm 135b in the configuration 550b of FIG. 5B, the interior or exterior models and corresponding virtual elements may be updated to reflect possible port placements.

Thus, in FIG. 5B, a portion 520a of the interior model 570a has now been updated (shown in updated version 570b of the model 570a) to reflect the region 520a (rendered as a virtual element surface, as a virtual element change in the rendering of the interior model texture, etc.) of the interior model upon which a port will be accessible to the arm 135b and its presently attached surgical instrument. As will be discussed, the dimensions of region 520a may be a consequence both of the arm 135b's articulation range, as well as the nature of the instrument attached thereto. In some embodiments, a team member may adjust the insertion axis of an instrument, which will update the overlays and other elements inside the patient body. In some embodiments, not only arm and instrument ranges of motion, but the relative location of other surgical equipment may be noted via virtual element renderings (e.g., arrows) within the interior model, e.g.: the location of a surgical cart, the location of a robotic system, the location of an instrument, positions of team members in the past, the present or anticipated positions in the future, etc., such renderings being particularly helpful to the surgeons 105a, 105c, whose attention is directed to the perspective of the current imaging device pose.

The exterior model 515a may likewise be updated to the updated form 580a (again, shown with an enlarged form 580b for the reader's convenience), which includes an exterior region 520d (again, possibly rendered as a virtual element surface) corresponding to the interior region 520a, which may be likewise highlighted, outlined, or otherwise suitably identified to the team members. In some embodiments, an outer range indication 520c may also be rendered to assist the team members with understanding the possible orientations (and consequent potential risks of collision) of arm 135b and its instrument relative to the exterior region 520d. As will be discussed herein, the outer range indication 520c may identify various approach angles with which the team may access the region 520d.

By alternating between, or by overlaying translucent renderings of both the interior model 570b and the exterior model 520a, team members may readily recognize relations between, e.g., the target anatomy and the accessible portions 520a, 520d, the accessible portions 520a, 520d and the possible limits and ranges of arm 135b (e.g., via indication 520c), as well as relations between these items and the current orientation of the imaging device-bearing instrument and other surgical elements, etc.

To further facilitate the reader's understanding, FIG. 5C shows another set of correspondences when team members consider potential port placements for the arm 135c rather than arm 135b (again, though the arm is shown moved in the configuration 550c, one will appreciate that different arms may be considered in some embodiments without a team member explicitly repositioning the arm). Again, the interior model is updated to the form 570c which includes an indication of the region 520b of the surface of the model accessible to the instrument of arm 135c. Similarly, the external model is updated to the form 590a, and the enlarged view 590b, to indicate a corresponding region 530d and corresponding outer range indication 530c for arm 135c and its instrument. Though individual arms are shown here for the reader's consideration, one will appreciate that in some embodiments the regions for more than one arm may be shown simultaneously relative to internal or external models for the surgical team's consideration. Indeed, in some embodiments, all available arms may be shown at once, the regions colored differently to correspond with their respective arm and instrument ranges. Intersecting regions, or choices precipitating collisions with arms or other devices, may also be highlighted.

Visualizations facilitating internal-external correspondences, as shown in FIGS. 5B and 5C, may help the team to identify a best approach for addressing the target anatomy in downstream tasks, while mindful of the wider surgical theater context. A computer system may adjust regions 520a, 520d and 520b, 530d as well as ranges 520c and 530c based upon port placement guidelines for specific procedures (e.g. linear placement, placement at certain distances, etc.) as discussed in greater detail herein. These guides may help surgeons to avoid “guesstimating” linearity and distance during port placement, a practice which can be especially inaccurate when confronted with a patient anatomy or surgical configuration new to the surgeon. Likewise, these guides may facilitate greater consistency in port placements across procedures.

### Example Arm and Instrument Range Representations

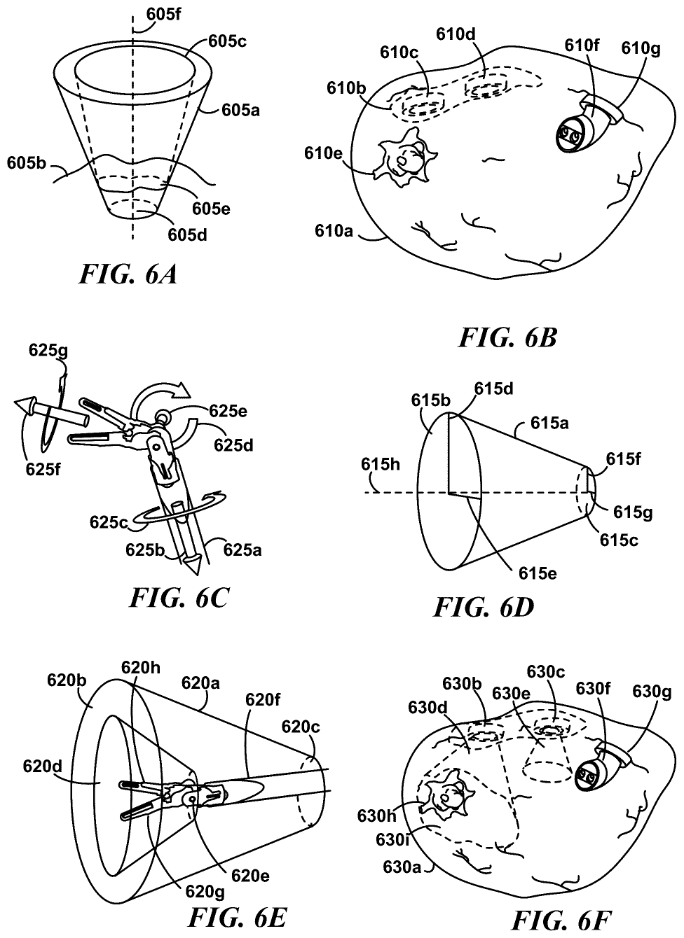

To facilitate the reader's comprehension of some methods for achieving the renderings of outer range indications 520c and 530c, FIG. 6A is a schematic view of an outer range indication virtual element, as may be implemented some embodiments. Specifically, a computer system may initially consider the full range of a robotic arm and its instrument as a geometric object (e.g., as a convex hull around the arm or instrument's outermost points of articulation), in this example represented by a conical section 605a (for clarity, one will appreciate that a conical section may be a cone tapering to a point, or a capped cone, as depicted here and in, e.g., FIGS. 6D, 6E, etc.). While the conical, cylindrical, etc., section 605a may extend through the surface of the patient 605b, the portion intersecting the interior 605e may be used to identify the regions 520a, 520d and 520b, 530d. Thus, the ranges 520c and 530c may be formed from the geometry 605a with the region 605d within the patient interior 605b removed (resulting in the new limit of the geometry corresponding to the portion 605e upon the patient surface 605b or upon the interior model).

The set of physically possible approach angles and arm/instrument poses resulting in intersection with the patient interior may not be the same as the set of viable approach angles and arm/instrument poses for a given surgical plan, patient anatomy, or instrument choice. For example, angles of approach outside a threshold angle from an axis 605f perpendicular to the surface 605b at the center of the region 605e, while associated with physically possible points of insertion, may occur at approach angles so nearly parallel with the patient surface 605b that they are not viable for the selected instrument. Consequently, the system may adjust the geometry to the acceptable limits, creating the revised geometry 605c. Such adjustments may be effected by rule sets determined by manufacturers and surgical teams in past procedures, e.g., as discussed herein.

Some embodiments may provide location information for a possible or recommended port placement. For example, FIG. 6B is a schematic view of a patient interior model 610a with example port placement recommendation representations 610c and 610d (e.g., virtual element surfaces, changes in texture to the interior model rendering, virtual element renderings of portal entries, virtual element tori, etc.), as may be implemented in some embodiments. Again, one will appreciate that the initial port placement rendering 610g and imaging device-bearing instrument depiction 610f may not be included in renderings of the interior model in some embodiments. A representation of at least initial port placement rendering 610g, possibly in combination with arrow 505d, may help viewers consider the relative location of port placement recommendation representations 610c and 610d to the imaging device-bearing instrument's present pose (and consequently, the operator's view). As mentioned, in some situations, the initial port placement rendering 610g may be the representation 370a as inferred from the kinematics of the port placement, since it may lie beyond the imaging device's visual range.

As previously discussed, the region 610b of interior model 610a may be highlighted, outlined, etc. or otherwise demarcated to indicate the accessible range of the arm and instrument (as were portions 520a and 520b). Within this accessible region 610b, the system may invite the user to select a specific port pose, e.g., one of port placement recommendation representations 610c and 610d. A user may select a location, e.g., by selecting the offered location, or where a continuous option is available, sliding a port selection icon along the surface of the interior model representation within the region 610b. In this manner, the viewer can consider the relative position of the proposed port placement to the target anatomy 610e, as well as to other proposed port locations, the location the imaging device-bearing instrument, etc. Once this more specific choice within region 610b is made, the system may consider the constraints of the corresponding approach angle, rule sets, etc., to, e.g., generate revised geometry 605c, if this was not done already. Revised geometry 605c may also be produced by considering the limitations imposed by other arm and port selections. Similarly, users may define constraints upon geometry 605a, such as a minimum biting angle for approach, maximum distance to the target anatomy 610e, etc., thereby producing a revised geometry 605c.

Various embodiments may also consider the specific constraints of the contemplated instrument, or set of instruments, of the arm under consideration. For example, FIG. 6C is a schematic view of an example collection of instrument degrees of freedom as may inform the conical depictions in, e.g., FIGS. 6D and 6E in some embodiments. This particular instrument 625a may be moved in the directions represented by arrows 625b, 625e, 625f. Similarly, subject to various constraints, the instrument may be rotated around the axes represented by the arrows, e.g., in the direction 625c, 625d, 625g. Various embodiments may analyze the forward kinematics of all these possible motions to determine outer bounds to the instrument's motion relative to a potential port placement, e.g., one of placements 610c or 610d. Thus, one will appreciate that the renderings disclosed herein may be a function not only of the determined patient interior anatomy and the selected robotic arm's range, but also of the selected instrument's range for that arm. Team members may accordingly consider choices of instruments for a given port choice (noting, e.g., their variation in reach and range) in addition to the port's relation to the robotic arm.

While some embodiments may represent the outer limits of the instrument's reach (e.g., the instrument 625a) with a point cloud, or convex hull around the reachable points, some embodiments may instead represent the reach with a conic section 615a as shown in FIG. 6D. Specifically, a narrowed end 615c of the section may have dimensions 615f and 615g corresponding to the dimensions of the contemplated port aperture (e.g., one of representations 610c and 610d). The center axis 615h of the conic section may correspond with the center axis of the port aperture. Unlike the end 615c, the selection of the dimensions 615d and 615e of the opposing end 615b may be determined by the outer accessible limits of the instrument (e.g., following a forward kinematics analysis of the degrees of freedom in FIG. 6C). For example, the system may select the dimensions 615d and 615e and the length of the conic section along the axis 615h such that the conic section is the largest conic section falling entirely within the outer bounds determined from the forward kinematics analysis of the degrees of freedom in FIG. 6C (or, alternatively, the smallest conic section fully containing the outer bounds, an average between these largest and smallest conic sections, etc.).

While the conic section 615a may not always represent the entire physically possible range of motion of the instrument from a given port selection, it may instead provide a consistent, recognizable foundation for the team members with which to assess a variety of placement options. Where more granularity is desired, the convex hull virtual element or similar structure determined via forward kinematics may instead be rendered. In some embodiments, an intermediate approach may be applied, wherein various of the instrument's degrees of freedom are rendered with their constraints and some without.

For example, FIG. 6E is a schematic view of a composite augmented reality conical instrument range virtual element, as may be implemented in some embodiments. Specifically, a virtual element rendering 620f of the selected instrument may be provided with a first conical section 620a, and second conical section 620d (in some embodiments, when the real instrument is inserted, virtual element rendering 620f of the corresponding contemplated real instrument may appear in the augmented reality renderings to inform team members; in some embodiments, only the SLAM-derived capture of the real-world instrument may instead be rendered as part of the interior model). The first conical section 620a may have length and dimensions of its ends 620b, 620c the same as conic section 615a (ends 615b, 615c, respectively). However, in some embodiments, the first conical section 620a may instead represent the maximum translational insertion distances from the portal entry for the selected instrument (that is, an independent degree of freedom). The second conic section 620d here indicates the outer range which clamps 620g and 620h may rotate around attachment screw 620e (i.e., around the axis associated with arrow 625e). Again, one will appreciate that, as discussed with respect to the conic section 615a, in some embodiments the viewer may elect to replace the conic section with the corresponding forward kinematics point cloud, convex hull, etc. for a more detailed perspective. By providing a more granular conical breakdown and representation of the instrument's range of motion for specific degrees of freedom, team members can more readily assess the viability of an instrument, arm, and port selection as relates to anticipated task actions within the context of the particularly contemplated patient interior.

The above described conic sections may also provide visualization benefits facilitating quick and ready discernment of port viability and nonviability to team members, a feature which may be especially useful given the often time-sensitive nature of the surgical process. For example, FIG. 6F is a schematic view of a patient interior mesh model 630a with example port placement virtual element recommendation representations 630b, 630c (which may be the same as representations 610c and 610d), as well as corresponding augmented reality conical instrument range virtual elements 630d and 630e as may occur in some embodiments. Again, one will appreciate that, though shown here to facilitate the reader's understanding, representation of the imaging device-bearing instrument's current position 630f and portal entry 630g may or may not be included within the interior model.

In this example, the range of the volume 630e is not long enough to intersect the opposing side of the interior model. However, the volume 630d is long enough to pass through the opposing end, forming an intersecting surface 630i with the interior model 630a. Thus, rather than render the remainder of the volume 630d past the intersection, the surface 630i may be rendered as part of the volume 630d or, e.g., as a differently textured, outlined, or otherwise highlighted portion of the mesh model. In some embodiments, the color of the region 630i may be adjusted in accordance with the length of the range of the original volume 630d extending past the wall of the mesh model. For example, the excess range may be associated with a heat map so that the viewer can readily discern how much tolerance is permitted by the port selection. In this particular example, the port location 630b would be more suitable for accessing target anatomy 630h than the port location 630c since the resulting region 630i readily encompasses the target anatomy 630h. In some embodiments, if the surgeon can identify the target anatomy, the computer system may calculate the distance from the proposed port location to the target anatomy (or from the periphery, or points within, region 610b) for different instruments and provide recommendations for instrument selection and port placement before presenting the more explicit rendering of FIG. 6F. In some embodiments, the system or users may be able to guide renderings of the selected instrument virtual element (e.g., element 620f) within the range volume, e.g., as part of an animation, to “role play” an upcoming or contemplated task action.

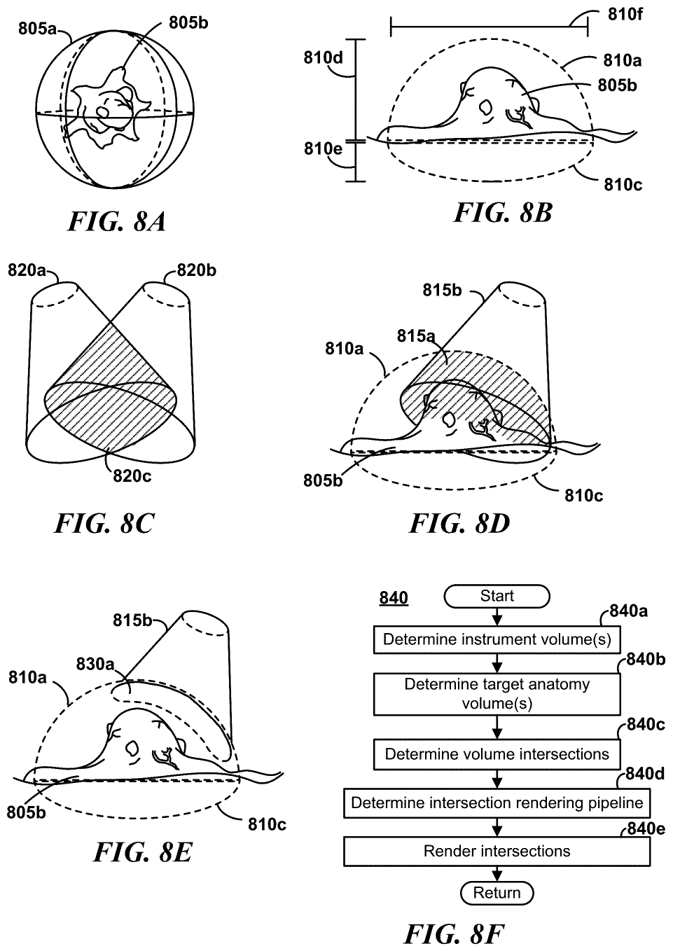

In some situations, multiple instruments may be used together, e.g., a needle driver and a grasper may be used together to perform suturing (e.g., in a two-handed task). Regions in which these instruments' respective ranges of motion intersect may likewise by highlighted for the user's confirmation, as will be discussed in greater detail herein with respect to FIG. 8C (e.g., that all the selected portions and instruments are able to adequately interface with region 630i, with target anatomy 630h, and with each other). Again, one will appreciate that particular procedures and instruments may have unique constraints and requirements informing the heatmap range, conic volume dimensions, etc. (e.g., an excision task may require greater tolerances than a suturing or inspection task, such tolerances specified in the rules accompanying the surgical plan).

For clarity, though port placement virtual element recommendation representations 630b, 630c, as well as the region 610b, are shown in the depicted examples as appearing upon the surface of the interior model, one will appreciate that since the imaging device's pose may be inferred from corresponding kinematics, and consequently the pose of the interior model may be determined relative to the surgical theater, representations 630b, 630c, region 610b, corresponding instrument range virtual elements, etc. may be rendered at appropriate locations even when those locations do not appear upon the interior model (e.g., where the model is incomplete, or when a local, live interior model is rendered). Thus, in some embodiments, port placement recommendations may sometimes appear in unobserved regions of the patient interior, e.g., region 340c.

### Example Arm and Instrument Range Representations—Example Rendering Processes

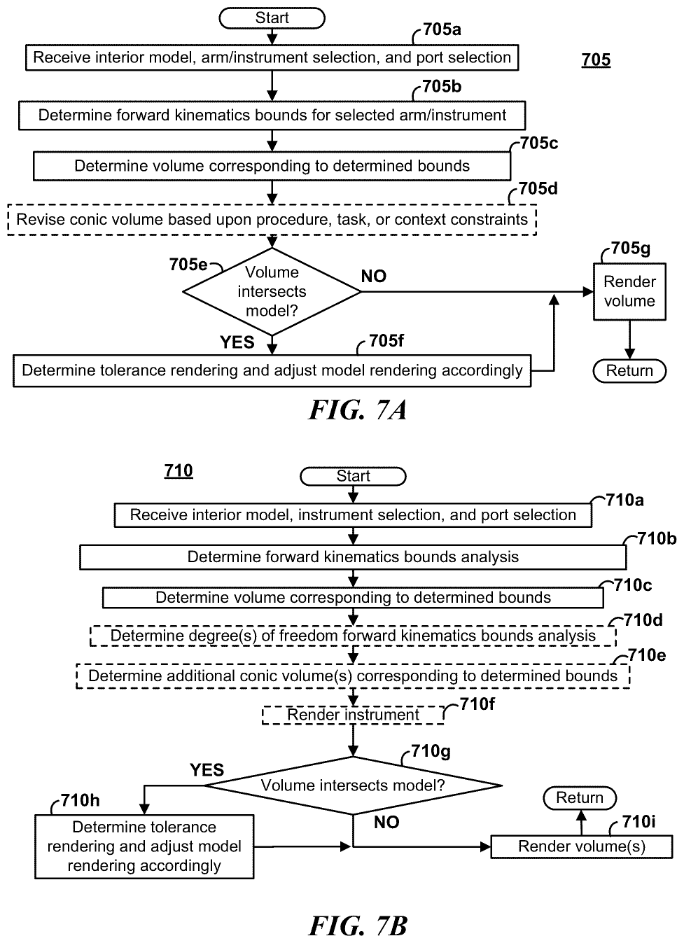

FIG. 7A is a flow diagram illustrating various operations in an example process 705 for rendering an external augmented reality robotic arm range representation (e.g., as shown in FIG. 6A), as may be implemented in some embodiments. At block 705a, the computer system (e.g., system 190b or system 190a in a non-robotic context) may receive the interior model, the current instrument selection, and the current port selection from one or more members of the surgical team, or from a computer system. At block 705b, the computer system may determine the forward kinematic bounds of the selected robotic arm with the selected instrument (in some situations, the influence of the instrument may be negligible, or excluded, and so only the arm's degrees of freedom may be considered). At block 705c, the system may then determine the volume, such as a conic volume, corresponding to the bounds determined at block 705b. As discussed above, the conic volume may be the largest conic volume falling within the kinematics boundaries (or the smallest conic volume encompassing the kinematics boundaries).

In some embodiments, at block 705d, the system may revise the conic volume based upon the constraints or objects in the procedure, task, or rule sets for the selected configuration, etc. For example, constraints imposed by the chosen instrument, the performed task, personnel or equipment locations, etc. may be introduced (e.g., creating the revised geometry 605c). At block 705e, the system may determine if the instrument volume intersects the model (e.g., producing portion intersecting the interior 605e). If so, at block 705f, the system may determine the degree to which the volume exceeds the model surface, or patient surface, and determine a corresponding tolerance rendering (which may appear, e.g., as a colored region virtual element upon the surface of the patient). The volume may include both the arm's range of motion and the attached instrument's range of motion so that the depth of this tolerance can be fully expressed. At block 705g, the system may render the conic volume, e.g., in the various augmented reality devices in the theater. If a tolerance rendering was determined for an intersecting surface at block 705f, the system may adjust the rendering of the portion of the interior model surface as well.

FIG. 7B is a flow diagram illustrating various operations in an example process 710 for rendering an internal augmented reality instrument range representation (e.g., the range representations of FIG. 6D or FIG. 6E), as may be implemented in some embodiments. At block 710a, the system may receive the interior model, instrument selection, and port selection. At block 710b, the computer system may determine the initial degrees of freedom for a forward kinematics bound analysis of the instrument's range of motion (e.g., in connection with a proposed port placement within an interior model). As discussed above, these boundaries may include the translational length of the instrument from the portal entry. At block 710c, the system may then determine a volume, such as a conical volume, corresponding to the determined bounds. Again, the conic volume may be, e.g., the largest conic volume falling within the kinematics boundaries and whose central axis is aligned with the center of the portal entry or the smallest conic volume encompassing the kinematics boundaries, similarly aligned.

In implementations rendering only the conic virtual element depicting the instrument's overall boundary range of motion analysis (e.g., the element of cone 615a in FIG. 6D), the process may proceed from block 710c to block 710g. However, where a depiction of the instrument itself is to be rendered (e.g., the virtual element 620f), the instrument virtual element may be rendered at block 710f. Similarly, where specific degrees of freedom (e.g., those selected by the viewer) of the instrument are to be considered, at block 710d the system may perform additional localized kinematics bound analyses for those degrees of freedom and determine corresponding volumes at block 710e (e.g., the conic volume 620d in addition to volume 620a of FIG. 6E). One will appreciate that the local bounds may be determined, at least in part, based upon the manufacturer's specifications, and may be recalled here from storage and transformed to the current interior model coordinates.

At block 710g, the system may determine if the volumes (e.g., the volume determined at block 710c) intersect the interior model's surface (e.g., the intersecting surface 630i). Where such an intersection occurs, then at block 710g the system may adjust the interior model rendering accordingly (or provide a supplemental virtual element identifying the intersection) at block 710h, e.g., adjusting the rendering in accordance with a heatmap corresponding to the amount of tolerance. At block 710i the system may then cause the volumes determined at blocks 710c, 710e, representations from block 710f, and any supplemental elements from block 710h to be rendered (e.g., providing the elements to a graphical rendering pipeline).

### Example Arm and Instrument Range Representations—Target Anatomy Visualization Supplements

In some embodiments, various orienting virtual elements, e.g., within the surgical console field of view, or in renderings of the interior model at various augmented reality devices, may be provided specifically for the target anatomy. For example, FIG. 8A is a schematic representation of a target anatomy 805b with a corresponding spherical virtual element 805a, as may be implemented in some embodiments. In this example, the virtual element is a sphere 805a, centered at the center of mass of the target anatomy and with a radius extending to the furthest peripheral position of the anatomy. One will appreciate that convex hulls around the target anatomy's outer bounds and other geometric shapes may likewise be used. A wireframe, billboard boundary, translucent rendering, etc. of the element 805a may facilitate ready identification of the target anatomy from a variety of perspectives within the theater. In some embodiments, the system may adjust the surface texture renderings associated with the target anatomy to call attention to portions of the anatomy (e.g., those accessible, or those inaccessible, to an instrument at a proposed port location). Elements such as spherical element 805a may help identify the approach angle of various instruments, and facilitate communication of the same between team members, during the procedure.

Relatedly, FIG. 8B is a schematic representation of the target anatomy 805b, but with a corresponding composite virtual element having an upper 810a region (above the surface of the interior model) and a lower region 810c (below the surface of the interior model). As with the spherical element 805a, or a hemispherical, element, the upper region 810a may provide a common reference for team members regarding the relative angles of approach to the target anatomy by various instruments. In contrast, the lower 810c region may help inform the tolerance associated with various tasks in connection with the target anatomy 805b and may, e.g., include data from a CT or other prior scan of the patient. For example, where the task is to excise the target anatomy 805b, or to biopsy a region of the anatomy 805b, the depth 810e of the region 810c may correspond to a minimum desired range of excision or biopsy. Similarly, the dimensions 810d and 810f of the upper region 810a may correspond to desired angles of attack by a contemplated instrument to the target anatomy 805b.

As discussed above, where multiple potential port placements are being considered, multiple boundary range volumes (e.g., conical volumes, convex hulls, etc.) may be rendered within the interior model and their intersection highlighted to call attention the presence, or lack, of sufficient overlap. For example, FIG. 8C is a schematic representation of two intersecting virtual conic volume elements 820a, 820b corresponding to ranges of motion for two instruments relative to their respective proposed port entries. Though not shown in this example, one will appreciate that if the volumes 820a, 820b intersect a wall of the interior model, they may be accordingly truncated (e.g., as in intersecting surface 630i, and, as discussed, a heatmap render of the wall may be adjusted). Here, the overlap representation includes a geometry element 820c representing the region of intersection of the volumes 820a, 820b. The intersecting geometry virtual element 820c may be rendered as a wireframe, billboard outline, translucent solid, etc.

Such intersecting representations may also be used to confirm suitable boundary ranges with respect to a target anatomy. For example, FIG. 8D is a schematic representation of the target anatomy 805b of FIGS. 8A and 8B, as well as a virtual element geometry 815a indicating the intersection of the instrument port placement volume 815b with the upper region 810a. Since the volume 815b does not fully encompass the lower region 810c, the port selection or instrument selection may not be suitable for the planned task operation upon target anatomy 805b.

In some embodiments, a user may be able to toggle between multiple modes of intersection rendering. For example, while volumetric intersections may inform the suitability of various port and instrument selections, particularly their ability to fully reach the target anatomy in a desired fashion, it may be difficult to infer the corresponding angles of attack for each instrument alone, or in combination. Thus, in FIG. 8E instead of rendering the intersecting volume 815a, the system may highlight only the intersecting surface 830a of the volume 815a with the upper region 810a. Intersecting surfaces between instrument conic volumes within region 815a, with sphere 805a, etc. may be rendered in differently colored outlines in this manner to more readily indicate the angle of attack of the instrument relative to the target anatomy, which may in turn better indicate the relation of the imaging device bearing instrument and the surgeon's field of view to the instruments and target anatomy when working with the instruments.

FIG. 8F is a flow diagram illustrating various operations in an example intersection rendering process 840, as may be implemented in some embodiments. At block 840a, the system may consider the various instrument virtual element boundary range volumes in connection with various instrument and port entry selections. At block 840b, the system may likewise determine any virtual element volumes associated with the target anatomy (e.g., those specified by the surgeon following inspection of the target anatomy). At block 840c, the system may determine the volumetric intersections of each of the volumes determined at blocks 840a and 840b. Where desired, the system may likewise determine surfaces of intersection at block 840c, e.g., as discussed with respect to FIG. 8E. At block 840d the system may determine the appropriate intersection rendering pipeline. For example, it may be confusing to simply render all the intersecting volumes and surfaces at once. Instead, the system may solicit a priority ordering from the user, or impose one itself. In this manner, individual intersections may be rendered only one at a time or multiple intersections may be rendered. Similarly, only intersection surfaces may be rendered with different colors or opacities than other of the volumes or surfaces, or otherwise rendered in a different manner, for clarity (e.g., as a billboard outline rather than as a surface texture or three-dimensional form). At block 840e, the system may then render the determined intersections in accordance with the parameters determined at block 840d.

Example Data Registration—CT/MRI/Etc. And Interior Model Reconciliations

In some embodiments, if a three-dimensional virtual element model of the target anatomy is available (e.g. a model of a kidney that contains a tumor, ureters, vasculature, etc.), the model may be registered onto, or at least relative to, the interior model as a single virtual element or as a composite grouping of virtual elements. Landmarks, such as a pelvis may also be used to facilitate relative alignment. Such registrations may also be facilitated by comparing sensor results, e.g., coordinating a CT scan or MRI scan with an ultrasound image. For example, ultrasound may be used to detect a kidney or other target anatomy's boundaries (e.g., the boundaries of a tumor). This information can be used for automatic or manual registration and reconciliation of the interior model pose, CT or MRI scan pose, etc. Once registered, the user may have a complete view of the target anatomy under the tissue surface, as well as the tissue surface (e.g., as discussed in FIGS. 5A-C). For example, the 3D reconstructed CT scan, MRI scan, ultrasound, etc. model and the interior model may be each overlaid upon the surgical field in the augmented reality rendering.

Such integrated views may be especially informative for port placement, facilitating the introduction of additional ports at appropriate locations to easily access the target anatomy and other important anatomical structures. Fluorescence imaging (e.g., per the Intuitive Surgical, Inc. Firefly™ methodology), other fluid injections, etc. previously only visible from the surgeon console point of view, may also be rendered as virtual elements in virtual and augmented reality representations (e.g., as their own virtual elements, as texture adjustments, etc.), thereby informing the entire team of the state of the surgery. Similarly, one will appreciate that team members may request rendering of only specific virtual elements (e.g., only the registered CT scan, only the interior model, only an instrument range volume, etc.), or rendering of other of the elements at a lower opacity, or different color.

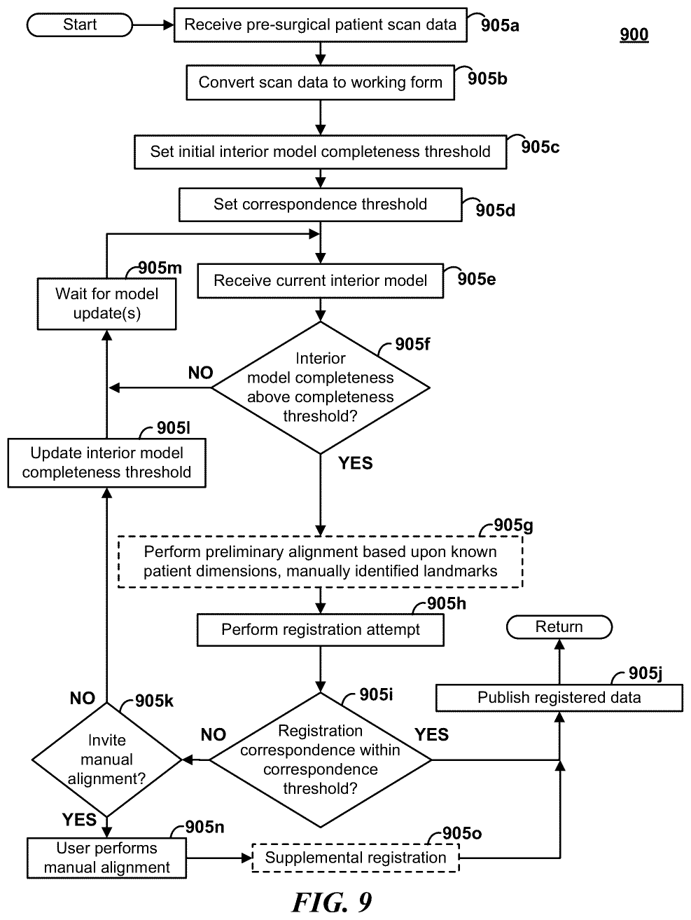

For example, FIG. 9 is a flow diagram illustrating various operations in an example process 900 for registering historical data (e.g., relative to an interior model), such as computed tomography (CT) data previously acquired for the patient, as may be implemented in some embodiments. At block 905a, the computer system may receive the pre-surgical patient data, e.g., the CT scans, ultrasound, etc. as discussed herein. At block 905b, the system may convert this material into a form suitable for comparison and integration with the interior model (e.g., following registration, the data may be converted to a TSDF or mesh format, may be overlaid upon the interior model, etc.). At block 905c, the system may set an initial value for a model completion threshold variable. For example, assuming the general dimensions of the patient (e.g., based upon the data acquired at block 905d) the system may infer the dimensions of the patient interior of the surgical site, and consequently, the relative completeness of the interior model. In some embodiments, the texture of the model, or its mesh, may be analyzed to determine the extent of completion (acknowledging, as discussed herein, that region 340c may not be expected to be completed in some implementations). For example, the ratio of complete to incomplete surface areas may inform the percent completion. Again, for clarity, in contrast to the depicted example, a “local, live” interior model may be assumed to be complete so long as the most recent depth data captures are within a desired temporal threshold. Alternatively, while the “local, live” interior model may be rendered, a stored copy of the full interior model integration results may be retained for use in the registration process 900.

At block 905d, the system may determine the corresponding threshold between the interior model and the prior data of block 905a. For example, the correspondence threshold may be the maximum acceptable cumulative error between points in the model and in the prior data following registration as when, e.g., a particle filter, 2D/3D deformation registration, etc. is used to determine the appropriate relative orientations of the prior data and the interior model.

At block 905e, during the surgery, the system may receive the interior model in its present state. At block 905f, the system may determine whether the interior model is sufficiently complete for registration with the scan, e.g., that its completeness value exceeds the threshold determined at block 905d. Where the model is expected to be sufficiently complete, then, in some embodiments, at block 905g the system may attempt an initial, preliminary alignment to assist the registration algorithm. For example, knowing the dimensions of the patient, the pose of the scanner producing the scan, and the pose of the imaging device, an initial registration estimate may be performed. As another example, the prior data may be scaled to assume similar dimensions to the model in anticipation of a more fully refined registration.

At block 905h, the system may then attempt to register the prior data and the model, finding a pose for the prior data or model resulting in the best alignment of their data. For example, one may apply Monte Carlo methods, such as a particle filter, to search the pose spaces for the match minimizing the distances between the most points between the prior data and the model.

At block 905i, the system may verify that the “best” identified pose of the model or for the prior data produces an error below the correspondence threshold determined at block 905d. Note that, in some embodiments, the correspondence threshold may also be adjusted in accordance with the completeness threshold, since one will often expect more matching points when applying a more complete interior model. Again, for clarity, in some embodiments, choice of a “local, live” interior model or of an integrated interior model need not be mutually exclusive. For example, the system may begin with creation and registration of prior data for an integrated interior model as described in this example, but following achievement of such registration, transition to the “local, live” interior model updates (e.g., upon a team member's request; indeed a full integrated interior model may continue to be acquired, but only the local, live rendering presented within that specific team member's augmented reality device upon the member's request).

If the prior data and interior model adequately correspond, then the determined pose for the prior data may be published (e.g., provided to the user augmented reality device rendering pipelines, recorded in computer system 190a or 190b, etc.) at block 905j. However, if the model and the scan do not yet satisfactorily correspond at block 905i, then, in some embodiments (not shown in this figure) the system may transition directly to block 905l or block 905m. In the depicted example, however, the system may also allow users an opportunity to perform manual alignment at block 905k. For example, where the model is sufficiently large and the operator may visually confirm the correspondence with the instrument's imaging device, it may be faster, or more accurate, for the operator to perform the alignment at block 905n. The system may further refine the correspondence at block 905o, e.g., reducing the error, as when the user alignment at block 905n serves an analogous function to the preliminary alignment at block 905g (indeed, in some embodiments operators or other team members may be invited to perform the preliminary alignment of block 905g). In some embodiments, the completeness threshold may be updated at block 905l to provide the mapping process sufficient time to supplement the model with more information, thus potentially effecting a better pose determination at block 905h.

### Example Port Placement Recommendation Algorithms

As discussed, surgical efficiency and effectiveness often depend upon coordinated actions and understandings among the team members. Poorly selected port placements, for example, can derail many downstream tasks, and so team members must coordinate their efforts despite the disparate target anatomy locations occurring in herniotomies, colectomies, etc., variations in procedure steps among surgical procedures, varied and dynamic theater configurations, changing patient dynamics, etc. While additional ports may be placed following the initial imaging device insertion, additional port placement analysis may also be needed later, in the midst of procedure, e.g., as the patient has moved, as target anatomy can no longer be reached, as assistive ports for additional instruments are needed, etc.

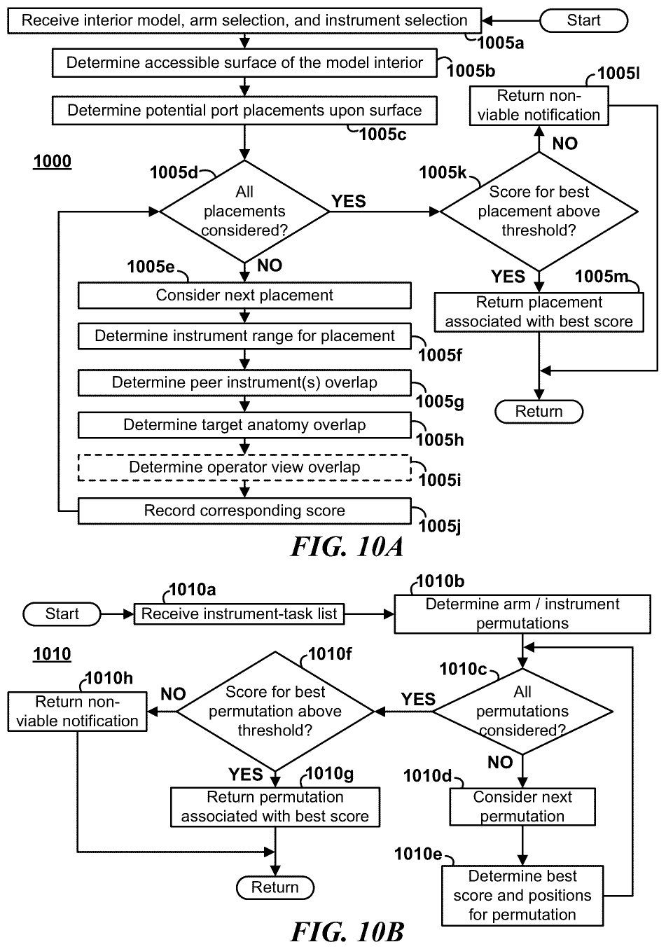

While operators and team members may select ports entirely at their own discretion, in some embodiments, the computer system may provide automated recommendations or guidelines. For example, FIG. 10A is a flow diagram illustrating various operations in an example process 1000 for specific (where the arm and instrument selection are provided) port placement recommendations, as may be implemented in some embodiments. At block 1005a, the computer system may receive the interior model, robotic arm selection and instrument selection for the robotic arm. At block 1005b, the system may determine the accessible surface of the model interior for the selected robotic arm, e.g., the portion 520a for arm 135b, or portion 520b for arm 135c.

At block 1005c, the system may then determine the potential port placements upon the surface within the region identified at block 1005b. For example, multiple placements may be produced by iteratively offsetting the placement throughout the region in discrete step sizes (the direction of the steps and their sizes chosen so as to cover the region). In some embodiments, Voronoi diagrams may be used, dividing the region into a number of segments (e.g., where the region's surface area is between five and six times the surface area of the portal entry, then a Voronoi division of 5 regions around 5 evenly distributed points may be performed). For example, FIG. 6B depicts two candidate portal entry placements 610c, 610d within the region 610b.

Once the system determines the candidate placements, the system may iterate through the candidates at blocks 1005d and 1005e. For the considered placement, the corresponding instrument range may be determined at block 1005f. For example, the system may determine the dimensions of the cone 615a corresponding to the instrument's reach relative to the proposed port placement, resulting in a geometry like the conical instrument range representation elements 630d and 630e. As indicated by the element 630d, overlap with a target region may be considered at block 1005h, including the tolerance available for the instrument beyond the target anatomy. As discussed with respect to FIG. 8C, if other instruments are being considered on other robotic arms at other port locations, then the system may also determine the respective overlap of their ranges at block 1005g (e.g., the region 820c). Some embodiments may also increase the score of the proposed placement when the overlapping instrument regions additionally overlap with the target anatomy.

In some embodiments, the system may also score the proposed placement based upon the instrument range's relation to the proposed operator's field of view at block 1005i. For example, the instrument imaging device's field of view may be treated as another “instrument range” and its degree of overlap (analogous to region 820c) used to determine a viewer overlap score at block 1005i. This may help ensure that the proposed port will not only suffice for physically performing the desired operations, but will also be cognitively accessible to the operator.

At block 1005j, the system may record the score for the proposed placement. For example, the score for the proposed placement may be the sum of each of the individual scores determined at blocks 1005g, 1005h, and 1005i. In some embodiments, the volumes of the overlaps may themselves be used as the scoring metric. The additional tolerance at block 1005h may also be construed as a volume for inclusion in the scoring metric. However, one will appreciate that different tasks may prioritize different of the volumes. For example, during an excision operation, the additional tolerance at block 1005h may be multiplied by an additional factor to give its successful presence more weight, since the excision should ideally result in complete removal of the target anatomy. Similarly, the score associated with the field of view in block 1005i may be scaled down where operator visibility is less relevant. The scores for individual instruments may likewise be up or downscaled at block 1005g depending upon their relevance to the task in connection with the instrument identified at block 1005a (e.g., range overlap may be more relevant to two suturing tools, which will be used closely with one another, as compared to a cauterizer and an irrigation tool, which may not need to necessarily operate at the same location).

Once all the candidate placements within the region have been considered, at block 1005k, the system may verify that the best scoring candidate (e.g., with the highest score) is viable for the planned task. For example, a poorly chosen arm may result in nothing but poor candidate scores, and the fact that one candidate placement's score is negligibly higher than the others should not result in the team believing that the arm is a viable choice. Thus, e.g., a minimum score may be a requirement at block 1005k and failure of all the candidate placements to satisfy that requirement may result in an indication at block 1005l that no viable port placement exists for this arm. Conversely, satisfaction of this verification may result in publication of the best scoring placement candidate at block 1005m. In some embodiments, a set of the best scoring candidates may instead be returned to provide the team with a choice of options.

While processes like process 1000 of FIG. 10A may be applied “on-demand” by members of a team when considering a specific arm, such processes may also be used as part of a wider evaluation of options available within the surgical theater (e.g., as discussed herein with respect to FIGS. 12 and 17). For example, FIG. 10B is a flow diagram illustrating various operations in an example process 1010 for general port placement recommendations, as may be implemented in some embodiments. In this process 1010, rather than considering a specific arm and instrument candidate, the team may wish to consider the placements within the wider context of the surgery and upcoming tasks. Thus, consideration of all the available arms and their possible instrument combinations may be helpful.

Accordingly, at block 1010a the system may receive an instrument-task list (e.g., as part of a surgical plan), which indicates tasks planned for the surgery and instruments that will be required for each. At block 1010b, the system may then generate all the possible permutations of arm and corresponding instrument choices based upon the list. At blocks 1010c and 1010d, the system may iterate through all the permutations identified at block 1010b and consider the best score for the permutation, including the best port selections for each arm and instrument. For example, at block 1010e the system may apply the process 1000 to each instrument's arm of the permutation, determining a most viable port placement (e.g., at block 1005m) for each arm, and then summing the scores for these most viable placements to determine a cumulative score for the permutation as a whole. In some embodiments, block 1010e may also consider the ordering of the tasks in its scoring. For example, if two tasks can be performed in succession using a same viable permutation choice, the benefit of reusing the existing arrangement may be preferable, even if a different permutation for the second task received a higher score. In these situations, a bonus scoring value, or scaling value may be applied, to recognize the benefit of minimal port and arm adjustments across tasks. Thus, in some embodiments, the number of instrument and port changes performed across all the tasks may also be used as a metric to inform the choice of permutation for any one task. Rules and prior surgical histories may also inform the scoring of a permutation, e.g., in accordance with a hierarchy of constraints. Thus, even the “exact same” port placement permutation may result in different scores if the surrounding theater context has changed.

Once the system has considered all of the permutations, then the system may verify that the best scoring permutation is in fact viable at block 1010f, e.g., that the best permutation's score is above a threshold (again, in some embodiments, a group of the best scoring permutations may instead be provided to the user for their selection). Where the permutation is viable, the system may identify the permutation and its scoring results at block 1010g. Conversely, notification that even the “best” permutation was not viable at block 1010h may inform the team that there exists a blocking constraint in their selection of tools, tasks, etc.

### Augmented Reality Assisted Surgical Team Coordination Overview

By providing an intuitive, common reference for the team members, various of the embodiments facilitate task coordination, e.g., port placements, whether the team chooses to perform the task entirely at their own discretion (using, e.g., the various visualizations disclosed herein), or in conjunction with computer-assisted recommendations, such as the port placement recommendations disclosed herein. For example, FIG. 11A is a schematic representation of a surgical robotic system with personnel employing various augmented reality systems, as may be implemented in some embodiments. In this example, as in the single imaging device insertion of FIG. 3A, the team has begun their procedure following sufflation by inserting only a single imaging device via arm 135d (again, port placement recommendations and other coordinated actions may also occur later in a surgery than immediately following sufflation). One or more of the members may wear or use augmented reality devices to perceive the virtual elements used to coordinate port placement and other theater operations. For example, the team member 1105c is wearing an augmented reality device 1105d, such as, e.g., a Microsoft HoloLens™, a Google Glass™ display, a Magic Leap 2 Headset™, etc. Similarly, the team member 1105e is viewing the augmented reality elements with an augmented reality enabled tablet 1105f. The touchscreen display on tablet 1105f (e.g., the display 1125a, as will be discussed) may enable team member 1105e to use finger gestures to manipulate the virtual elements for panning, zooming, rotating, repositioning, resizing etc. (e.g., as shown for virtual element model 1105b in FIG. 11A, but for member 1105c). As depicted in the figure, team members may also use gestures within their augmented reality device's field of view, handheld controls, etc., to perform various operations.

Though not shown in FIG. 11A, operator 105c and console 155 may also be present in the theater, or may be interacting remotely via telepresence. In some embodiments, operator 105c may cause the display 160a or another nearby display, to render the augmented reality perspective of members of the theater (e.g., to perceive both virtual elements internal or external to the patient). Conversely, in addition to possibly perceiving the view on the displays 125, 150 of an augmented reality device (e.g., one of headset 1105b or tablet 1105f), e.g., members 1105c and 1105e may cause their devices to render the perspective of the operator (e.g., as in FIG. 11A), e.g., as a billboard or planar surface in their field of view textured with the current instrument imaging device output (such a planar surface may be placed near the interior model for ready comparison by operator 105c and members 1105c, 1105e). Remote observers may also perceive the imaging device and augmented reality views via telepresence, as well as may participate in preparing the surgical plan, as discussed herein. In some embodiments, however, robotic control may be transferred to a team member's gesturing, and so the console 155 may be removed or temporarily superseded (e.g., team member 1105e may be controlling an instrument within the patient by hand gesture). Similarly, though robotic systems are discussed extensively here, augmented and virtual reality elements, port placement recommendations, planning, etc., may be applied, mutatis mutandis, in the non-robotic theater 100a.

As shown, and as will be described in greater detail herein, the team member 1105e may gesture to interact with various of the augmented reality graphical elements. For example, finger gestures may be used for panning, zooming, etc., or for scaling, rotating, or translating virtual elements (or for returning them to their corresponding real-world position), as the element 1105b has been moved by member 1105c. Device 1105f may also include a touchscreen display enabling the user to manipulate virtual element renderings by panning, zooming, rotating, repositioning, resizing etc. using finger gestures. While useful for coordinating discussions in the general surgical theater, such embodiments may also be useful for pedagogical purposes (including discussing proposed port placements), facilitating real-time interaction between teachers and students inside and outside the surgical theater. For example, member 1105e may use the AR-glass's controller to point and mark/draw on the surface of the interior model, which can then be viewed by the surgeon console as well as by other users of displays, augmented reality devices, virtual reality devices, etc. Similarly, in some embodiments, the models 1105a, 1105b may include digital markers/fiducials drawn by the surgeons (e.g., drawn or placed by the surgeon within the view 1110a). Conversely, markers/fiducials placed, e.g., via gestures, by members 1105c, 1105e in models 1105a, 1105b may likewise be visible to the operator in the view 1110a (e.g., when the operator pans the imaging device to a field of view encompassing the position of the placed marker or fiducial). Thus, alternatively or in addition to the compass 1110c, such fiducials may readily facilitate coordination between the team members, teachers, students, etc., despite their various locations and orientations around the surgical theater or elsewhere.

In this example, the team members 1105c and 1105e are viewing the partially completed, or fully completed, interior model 1105a, and associated virtual elements, relative to the actual location of the patient within the theater. For example, a team member may adjust the insertion axis of an instrument, precipitating corresponding updates to overlays inside model 1105a, e.g., as described in FIGS. 5A-C. In this example, the interior model 1105a is displayed “translucently” within the patient's body so the team members can “see through” the stomach and perceive the rendered mesh in correspondence with its real-world location. Thus, the interior model 1105a may appear in a form analogous to an open surgery, but without requiring a top-down perspective only and with visibility even when there are intervening occluding elements in the theater (e.g., sheets, other team members, theater equipment, etc.). The ability to perceive the surgical procedure, independent of an imaging device's present pose, from a variety of angles, and despite occlusions, may facilitate both a common reference and a variety of perspectives for the team members.

As shown in FIG. 11A, the interior depiction may include a variety of virtual elements, e.g.: proposed portal placement positions, instrument ranges, an arrow indicating the imaging device's present pose, highlights for target anatomy, a compass for coordinating references in local or global coordinate systems, etc. In some embodiments, renderings of surgical instruments in their current or proposed positions may also appear within model 1105a. Where kinematics information is available for the instruments, one will appreciate that their current pose and location may be inferred and rendered without relying upon SLAM-based capture of their depth representation (though, in some embodiments, the SLAM-captured representation may instead be rendered). In some embodiments, portions of the model associated with general portions of the patient's body may be shown translucently, whereas various elements of interest (surgical instruments, proposed port placements and instrument ranges, the imaging device pose indication, overlap with the surgical field, overlap between instrument ranges, the target anatomy, etc.) may be rendered at full opacity or at a higher opacity, with an outline, etc. so as to more readily facilitate their visualization. In this manner, the elements within the patient's body significant to a particular action or role may be made more clearly visible to a specific team member.

While the model and its elements may be rendered in the same physical location as it appears in the real world, as in the model 1105a, in some embodiments, gestures by team members may allow them to translate, rotate, and scale the model or its elements, so as to be personally viewed or shared with the team. Here, for example, the team member 1105c has through gesture motions created a copy 1105b and a compass 1105g for reference of the augmented reality model 1105a, and translated the model copy 1105b to a location closer to their face (effectively enlarging the model for their inspection). Note that the renderings of models 1105a may be animated to reflect updates to the model creation, port recommendation, imaging device orientation, etc. as discussed herein, and such animations and updates may be simultaneously mirrored in the model copy 1105b. In some embodiments, collisions between instruments (whether presently occurring, anticipated, or indicating a historical collision) may receive their own augmented reality representation, such as a highlighted geometry (e.g., a bright red faceted surface), in conjunction with auditory indicia, to alert the team to the potentially undesirable intersection.

Various representations of equipment, personnel, etc. in contemplated or future poses within the theater may also be rendered, e.g., in accordance with the surgical plan or a current discussion among the team members. For example, the virtual equipment element 1105h may correspond to equipment that will be moved to the depicted location in a later task in accordance with the surgical plan. Team members may create, move, and adjust such elements as part of a “virtual dollhouse” to discuss various theater configurations. Similarly, proposed port placements may be considered without moving the actual robotic system to the patient, but by considering virtual rendering of the robot in a proposed position and configuration. Members may raise or lower robotic booms in this virtual rendering, perform targeting just as they can on the real robotic system, cycle through past cases via representations of their equipment in the corresponding locations at a given point in their surgeries, etc. Thus, the team may adjust virtual ports and spar poses to simulate and visualize the result within the theater before finalizing or updating their surgical plan, or before performing the contemplated action.

FIG. 11B is a schematic representation of the field of view 1110a within an operator console for a surgical robot (e.g., the console 155 or upon display 150 or display 125). Again, for clarity, the augmented reality views of the interior model may indicate the present orientation of the imaging device with a model of the instrument, an arrow, such as arrow 515c, etc. These virtual elements may correspond with the imaging device's pose and consequently with view 1110a. Other virtual elements appearing in the augmented reality devices may likewise appear in the view 1110a being, e.g., overlaid, or otherwise integrated with the live video feed from the imaging device.

Thus, the depiction of the target anatomy 1110b may or may not include virtual elements in view 1110a, being an actual imaging device view of the target anatomy for the present pose of the imaging device. Similarly, while the augmented reality depiction of a proposed port placement 1110d and associated instrument range of motion 1110e may also be visible to team member 1105c in the rendering 1105b and to team member 1105c in the rendering 1105c, the proposed placement 1110d is shown in view 1110a from the perspective of the imaging device in its present pose. A compass indication 1110c may be provided, indicating, e.g., local coordinates within the patient interior, coordinates relative to the imaging device's present pose, global coordinates relative to the entire theater, etc., so as to facilitate communication among the team members.

In contrast to the operator 105c's focus (or operator 105a) primarily upon the view 1110a and the patient interior, the team members 1105c, 1105e may more readily alternate between the interior model and exterior or theater wide augmented reality elements. To facilitate the reader's understanding, FIG. 11C is a sequence of images showing the state of an augmented reality depiction for exterior personnel within the theater, from a first perspective 1115, as may occur in some embodiments. Though reference is made herein to a tablet 1125a to facilitate the reader's understanding, one will appreciate that a corresponding field of view and representation may be achieved with other augmented related devices, such as a monocular or stereoscopic headset display, projecting device, etc. For example in some embodiments, the robotic system may include a projector displaying the model 1105a from a single perspective upon the patient's surface (analogous, e.g., to a top-down open surgery), upon a wall of the surgical theater, etc. Such projections may be useful where there are team members or observers who are not themselves equipped with augmented reality devices.

In this example, in the state 1115, before the team member raises the hand-held 1125b tablet 1125a to visualize the patient 120's anatomy, the system may render virtual elements depicting overall theater-context, surgical plan actions, etc. For example, an arrow 1125c, or other directional indicator, may direct the team member to their next action item in accordance with the surgical plan (e.g., preparing an instrument for insertion as part of an upcoming task, assuming a different position around the patient in anticipation of an upcoming maneuver, etc.). Similarly, textual display 1125d may provide a transcript for the member's reference of events and verbal team member statements occurring previously in the surgery, notes taken by team members, patient history data, etc. Textual display 1125d may also indicate the current, past, or future state of the surgical plan, providing the member with a general context of the state of surgery, e.g., in accordance with the team member's role (role or pose-based constraints upon visual element rendering may limit clutter in the interface).

When the member raises 1170a the device 1125a so that the surgical site appears within the device's field of view, new virtual elements may appear (though removed here for clarity, elements 1125c and 1125d may also remain in some embodiments). For example, where the member desires, or is designated by their role to view, the interior model, as in situation 1120, then the user may perceive the interior model virtual element 1125f, which may be the same as the model 1105a, with the same corresponding elements within it, but rotated, translated, and scaled so as to correspond with the member's current pose when holding the device 1125a. A compass 1125g may, e.g., indicate the relative orientation of the instrument imaging device to a global coordinate system represented by the compass 1125e.

In contrast, where the user selects a different view, or their role designates them for viewing a different view, the augmented reality elements may be correspondingly adjusted. For example, in anticipation of an upcoming port placement, in the situation 1125 the user instead perceives external augmented reality elements (e.g., in accordance with their role), such as an arm approach guide 1125h (e.g., one of ranges 520c and 530c or conical section 605a; as shown, guide 1125h may appear relative to the surface of the patient in some embodiments, without rendering the interior model, or its exterior surface, rather than relative to a rendering of the interior model or its exterior surface). In this manner, when considering one or more potential port placements, team members can review the internal model to verify that the internal conditions of the chosen arm and instrument will be satisfactory, as well as view external elements to ensure that the proposed placement will agree with external conditions in the theater (e.g., to avoid collisions, anticipate future placements, anticipate future instrument swaps, etc.). In the case of future instrument swaps for a given placement, multiple instrument ranges may be considered from the proposed location to ensure that each may readily perform its desired function in the upcoming tasks.

The correspondence between the surgeon console view 1110a, interior models 1105a, 1105b, and exterior model elements (e.g., range 1125h) may create a shared context for the care team to further interact and explore. The team may, e.g., iterate through different port placement strategies, visualizing the positioning of the instruments, their reach, and the size of the surgical field. Insights on the number and type of instrument collisions from past cases may also be noted (e.g., in the textual display 1125d) pertaining to a certain port configuration. The surgeon may change the proposed port position dynamically (e.g., sliding the port along the surface of the interior model in a region 610b) and visualize the changes in the surgical field.

For example, while the surgeon is focused on the visualizations inside the patient anatomy, one of members 1105c, 1105e may focus upon the visualizations outside the patient body such as the positioning of the patient table, the final position of the patient cart, the cart's pose, and any possible arm collisions that have happened in the past. In addition to such real-time assessments, the team may also predict future states using virtual equipment representations. For example, upon the surgeon's request, one of members 1105c, 1105e may interact with a virtual model of theater equipment (e.g., like virtual equipment 1105h), such as the robotic cart, to change the instrument axis or imaging device view (which may then precipitate an update in the surgical field view during a simulation of the proposed change). Once the surgeon is comfortable with the port placement strategy, the computer system may broadcast the updated surgical plan to all the other care team members (e.g., as an update to the surgical task plan appearing in textual display 1125d). Where a port recommendation was based upon a planned location of the robotic system, following confirmation, one of members 1105c, 1105e may then use the final port selection to drive the robot cart to the desired location for the procedure. One of members 1105c, 1105e may then adjust the arms to the final pose, attach cannulas, an endoscope, other instruments, etc.

For further clarity, FIG. 11D is another sequence of images showing the state of an augmented reality depiction for exterior personnel within the theater, from a second perspective, as may occur in some embodiments. Again, in the situation 1130, the tablet 1125a has yet to be raised to view the surgical site of patient 120. Consequently, in this example, the tablet 1125a continues to render a text display 1130b with contextual information and a directional indicator 1130a of the team member's next scheduled action, pose, etc.

Once the tablet is raised 1170b so that its field of view encompasses the surgical site, various virtual elements may be rendered in accordance with the team member's present viewing configuration (which may, again, be a function of the member's role in the surgery). For example, in situation 1135 the member's device is configured to view the interior model virtual element and consequently a rendering 1150a of the model 1105a in the orientation relative to the member's current pose may be presented. Again, for clarity, each of the elements within the model, including the proposed port location, the imaging device orientation, etc. may be reoriented in accordance with the viewing pose of the member's augmented reality device. A compass 1150b may again help inform the member of their augmented device's, the theater's, the interior model's, the robot system's, etc., relative coordinates, thus facilitating ready communication between the team members.

Conversely, when the member's augmented reality device 1125a is configured to render external elements, as in situation 1140, the device may present the arm range indication virtual element 1155a (e.g., corresponding to the port location and instrument range shown within the interior model 1105a). For clarity, the arm range indication 1155a may be presented in isolation relative to the surface of the patient, without rendering the interior model (e.g., per the user's preference).

### Example Composite Elements and Virtual Surgical Planning Interface

As discussed with respect to FIGS. 11A-D, the augmented reality representation of the surgical environment may include a variety of virtual elements, such as virtual equipment 1105h, indicating a future location (contemplated by the team or expected in a surgical plan) of the equipment in an upcoming task, as well as directional indicators, like arrows 1125c or 1130a directing the user to, e.g., a pose or equipment associated in the next action in a current or upcoming task. In some embodiments, these virtual elements may be rendered as part of the surgical plan, which may be initially created, and possibly subsequently updated, using an interface including various of the schematically depicted graphical user interface elements of FIG. 12. Thus, surgical planning may be synchronous (before the procedure, at pre-planning block 205b) and asynchronous (during the procedure at the team's discretion, e.g., at block 205f). Specifically, in lieu of seeking to algorithmically anticipate adverse events in the surgical theater, some embodiments instead allow users to “roleplay” with virtual models of equipment, actions, etc. in virtual reality, augmented reality, or with an interface with graphical elements as shown in the interface 1200, or a combination of these rendering approaches. For example, it may be much more effective to move virtual element equipment model 1105h into position for an upcoming task and verify by manual visual inspection that the location will not present an obstacle to personnel or other equipment, before actually retrieving and placing the equipment corresponding to virtual equipment model 1105h or instead attempting automated detection of an upcoming collision with the equipment. Thus, elements of interface 1200 may appear, e.g., in a laptop, desktop computer, a tablet, etc. prior to the surgery, when planning, as well as in the augmented reality devices or displays (including the robotic console) of the theater, during the procedure.

Example graphical elements in schematic interface 1200 include a depiction in region 1205 of the interior model and a schematic representation in region 1210 of the theater using virtual element depictions. Before the surgery, the region 1205 may indicate an idealized version of the patient interior model based upon historical data or the user's initial placement. Similarly, the representation in region 1210 may be populated with virtual models of equipment, personnel, robotics system equipment, the patient, etc. (in some embodiments, only one of regions 1205 or 1210 may be presented). Roleplaying with these models may extend the operating team's “predictive horizon” for anticipating future task actions, adverse events, etc. In some embodiments, as pose registrations become updated throughout the procedure, the depictions in the representations of regions 1205 and 1210 may likewise be updated to reflect the poses and states of their real-world counterparts.

Thus, as will be discussed in greater detail with respect to FIG. 17, various embodiments may allow the user to place virtual elements (representing surgical equipment, personnel, the patient, etc.) and direct their behavior at various points of the surgery, thereby creating a surgical plan. During virtual execution of this plan, a current time indicator 1215c may advance along a timeline 1215a in region 1215 (e.g., during playback of a procedure or surgical plan via controls 1215b). In this example, seven planned tasks associated with the surgery are also indicated along the timeline 1215a. In this example, the interface 1200 is being used during the surgical operation, and Task 3 is highlighted to indicate that theater data indicates that the surgery is presently in Task 3. During a live surgery, in some embodiments, moving icon 1215c before the present time will result in population of regions 1205 and 1210 with virtual elements in accordance with the acquired real world data at that past time, whereas advancing indicator 1215c past the present time, may result in population of regions 1205 and 1210 with virtual elements in accordance with the anticipated state of the surgical plan at that time (rather than their real-world counterparts), either entirely in accordance with the virtual predictions or designations by the surgical planner, or supplemented based upon previously acquired real-world data from the current surgery (e.g., the interior model rendering 1205b may still reflect the result from the real-world data acquisition).

Here, the indicator 1215c appears at a planned time in the future of the surgical plan, during Task 5, whereas the highlight of Task 3 indicates that the surgery is presently in that task. Markers and other highlights may be used to indicate breaches in rules, near breaches in rules, the actual occurrence of adverse events, etc. Here, for example, there were two periods 1215e and 1215f indicating regions during which one or more rules were nearly broken (e.g., near arm collisions or poor fields of view). Selecting the periods in the timeline may present an explanation for the warning. Similarly, the region 1215i indicates a possible future period of near rule breaking (e.g., two instruments are expected to travel within an undesirable distance of one another; a port is planned for a historically poor location; etc.). Icons 1215g, 1215h, and 1215j may instead indicate time points when adverse events, such as collisions, are expected to occur if the current surgical plan is maintained.

As shown, region 1205 may include many of the virtual elements that will appear, or be presented, to team members in their augmented reality devices. Here, for example, a proposed port placement 1205c, virtual instrument rendering 1205g, and corresponding range depiction 1205f are provided. Similarly, the target anatomy 1205a as captured during the preceding portions of the surgery (e.g., in Task 1) may also be shown. A virtual element 1205d corresponding to the planned imaging device pose may be rendered. Coordinate system 1205e may likewise correspond to the local coordinates of the imaging device or the global coordinates of the theater, e.g., the robotics systems.

This rendering in region 1205 of the interior model may also correspond with the rendering 1210m of the model in region 1210. Thus, the arrow 1210f shown in model 1210m, as with an arrow in an augmented reality depiction, may correspond to the current or expected view of the imaging device 1205d, here, as attached to arm 1210n. Similarly, a local coordinate depiction 1210e may be provided as well as a global coordinate depiction 1210k (though shown here schematically, depictions 1205e and 1210k may indicate how the user's views of regions 1205 and 1210, respectively, each correspond to the common coordinate system of the theater, at least with respect to rotation; similarly, e.g., depiction 1210e may track depiction 1210k). Though not shown in this example, in some embodiments, the region 1205 may also depict virtual elements appearing outside the interior model (e.g., arm range indication 1155a).

Though shown here as a top-down view, region 1210 may facilitate viewport movement around the virtual theater rendering in three dimensions. As, e.g., a form of “smart dollhouse” or “virtual dollhouse”, virtual element assets and actions may appear in panel element 1220 (in some embodiments, placement of an asset in region 1210 may precipitate a corresponding virtual element in the in-theater augmented reality renderings, e.g., virtual equipment 1105h, during the operation). Some selected virtual elements may be placed by the user within representation 1205 or 1210 (and if desired, immediately appearing in the augmented reality renderings of the team members). For example, selecting an icon 1225a in the theater assets panel element 1225f presents assets representing personnel (e.g., one of operator 1210h, patient 1210d, first assistant 1210a, and second assistant 1210j) and the patient in various roles. The graphical editor may also facilitate editing the properties of various such virtual elements, as in specifying that first assistant 1210a will use an augmented reality headset 1210b and that second assistant 1210j will use an augmented reality tablet 1210i. Equipment, such as surgical bed 1210c, display cart (via icon 1225b with an example shown here as virtual element 1210p) and surgical trays (via icon 1225b, with an example shown here as virtual element 1210l) may also be generated. The icon 1225c may facilitate selection and placement of one of a variety of surgical robotic models, including the four armed unit represented here by virtual element 1210o (in this example, shown translucently in part so as to better view its arm orientations). Similarly, an icon 1225d may be provided for selection, configuration, and placement of other assets, such as console 1210g. Naturally, additional equipment may also be rendered, as represented by ellipsis 1225e.

Instruments upon the robot may be assigned per instruments panel element 1230a, via, e.g., icons 1230b, 1230c, representing an imaging device and cauterization instrument respectively. Again, ellipsis 1230d indicates the possibility of additional instrument types. Similarly, a task action panel element 1235d may show actions pertinent to the current task (in this example, Task 5, rather than Task 3, in accordance with the position of icon 1215c). Here for example, Task 5 includes an excision action 1235a and cauterization action 1235b (as well as others indicated by ellipses 1235c).

Choosing various combinations of actions and virtual element configurations may precipitate changes to the surgical plan, which may themselves be presented by additional icons in regions 1205 and 1210. For example, here, the excision action has been selected via icon 1235a, which will require attachment of a new instrument to one of the robot arms. Accessing this arm will itself require that the virtual cart 1210p be relocated. Thus, the user may specify a path plan 1210q for the personnel who will receive the role associated with the virtual member depiction 1210j. The path plan 1210q may, e.g., correspond to arrows 1125c and 1130a during the augmented reality rendering of the procedure (and in accordance with assistant 1210j's role, be visible only within the augmented reality device used by the member assuming that role), directing the team member around the robot as indicated so that the team member may begin moving the real world cart associated with the depiction 1210p to accommodate the instrument swap upon the robotic arm. In some embodiments, outer tolerance boundaries may be rendered around equipment, paths, like path 1210q, and other virtual elements (e.g., in accordance with the rules) to help avoid collisions (e.g., both in this interface and in the augmented reality viewings). Additionally, much like keyframes in animation, the user may specify various configurations and positions of the virtual elements in regions 1205 and 1210 at various times, and the system may then interpolate, applying rules and conditional logic, to verify the adequacy of the proposed and intervening theater states.

### Surgical Operations Planning-Role Based Data Siloing and Optimization

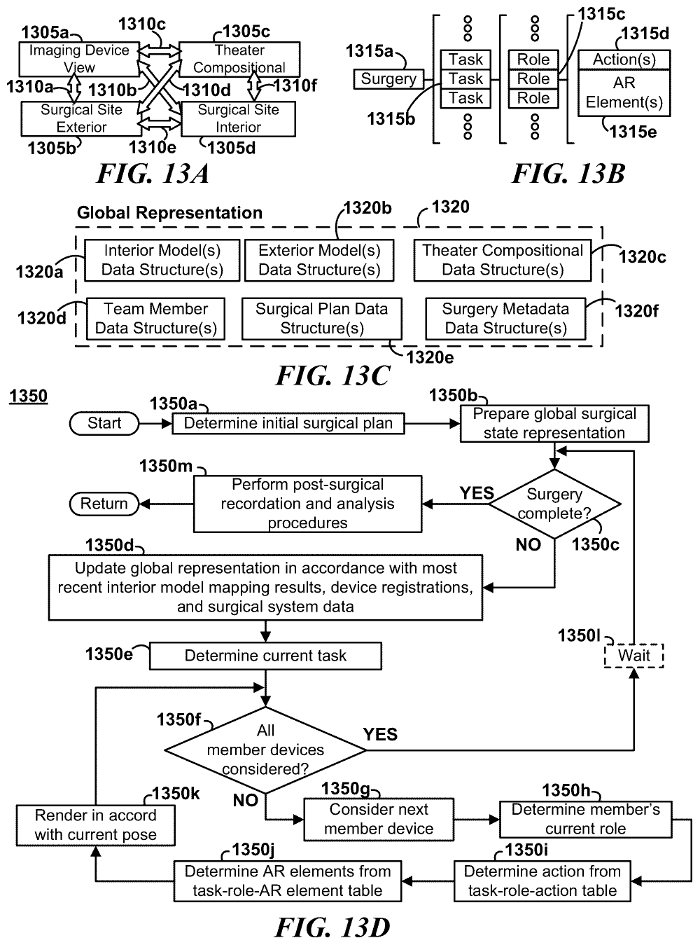

As discussed herein, various team members may acquire different augmented reality views of various elements within the surgical theater, not only in accordance with their respective poses within the theater, but also in accordance with their devices' respective configurations, such configurations possibly selected in accordance with the members' respective roles throughout the procedure. For example, FIG. 13A is a schematic block diagram illustrating various relations between surgeon console and theater-wide augmented reality depictions, as may be considered in various embodiments. In many embodiments, the collections of augmented reality elements may generally exist as one of four groupings: elements appearing within the imaging device view 1305a, as, e.g., in an endoscope eyepiece view or the surgeon console view (e.g., within the field of view 1110a); elements appearing exterior to the surgical site 1305b (e.g., range 530c and region 530d); elements appearing in the model interior or patient interior 1305d (e.g., the port placement recommendation depictions 630c, region 610b, arrow 515c, etc.); and elements appearing as a set of compositional elements 1305c for the wider theater surgical plan coordination (e.g., virtual reality depictions of surgical carts, equipment, robotic arms, etc., like virtual equipment 1105h). One will appreciate that correspondences may exist between these elements, sometimes resulting in the same element rendering, at other times resulting in a corresponding virtual element renderings. For example, the region 610b may appear both within the surgeon console as one of elements in grouping 1305a and model interior elements grouping 1305d, whereas the arrow 515c may appear explicitly in model interior 1305d to member 1105c, but only be implied by the current field of view in the surgeon console via grouping 1305a. Depending upon a team member's role during a portion of the surgery, the member may be directed to elements in any one of collections 1305a, 1305b, 1305c, or 1305d. Often, however, tasks may be associated with rules indicating which collections or which elements from which of collections 1305a, 1305b, 1305c, or 1305d are to be presented to a specific team member role. Such rules may include “global” rules relevant to the entire surgery (e.g., renderings all, or most, members perceive to avoid collisions), and “local” rules (e.g., rules to guide a member through their specific role during a task).

For example, rules 1310a may indicate how adjustments to imaging device view elements 1305a affect surgical exterior elements 1305b and vice versa (e.g., when an operator selects port location 630b rather than port location 630c, the corresponding range 530c may be adjusted to inform viewers of the external virtual elements of the new relevant arm range, and vice versa). Similarly, rules 1310b may indicate how adjustments to imaging device view elements 1305a affect surgical interior elements 1305d and vice versa (e.g., when an operator highlights anatomy 1110b within the view 1110a, then the rendering of the corresponding portion of the interior model 1105a may likewise be adjusted, and vice versa). Rules 1310c may indicate how adjustments to imaging device view elements 1305a affect theater compositional elements 1305c and vice versa (e.g., another team member's movement of a virtual or actual cart for an upcoming task may limit an arm range of motion and consequently eliminate one or more proposed port placements in the view 1110a). Rules 1310d may indicate how adjustments to the exterior elements 1305b affect compositional elements 1305c and vice versa (e.g., selection of an arm may present a range 530c, which may also result in warnings or limitations on virtual or actual cart placements in the theater). Rules 1310e may indicate how adjustments to the exterior elements 1305b affect interior model elements 1305d and vice versa (e.g., restriction of an arm range, as in FIG. 6A, may result in removal of various proposed port placements within the interior model). Finally, rules 1310f may indicate how adjustments to the theater compositional elements 1305c affect the interior model elements 1305d and vice versa (e.g., selection of a port placement in the interior model may block certain paths and equipment locations to accommodate the associated robotic arm).

While, as discussed above, some rules may facilitate coordinated rendering of virtual elements between the groups 1305a, 1305b, 1305c, 1305d to provide a common reference for all the members of the theater, some rules may also consider adjusting renderings in accordance with the role being presently performed by each team member. Accordingly, in some situations, some virtual elements may be visible only to some team members and not to others (e.g., instructions for robotic cart placement may be seen by assisting team members in the theater, but not by the operator 105c within the surgeon console 155).

For example, FIG. 13B is a schematic block diagram illustrating various relations between elements in task-role-action and task-role-AR element tables, as may be implemented in some embodiments. In this example structure, a surgery 1315a (including a surgical plan) may be decomposed into tasks as discussed herein. Each task, e.g. task 1315b, may itself include a number of roles for the various team members (surgical operator #1, anesthesiologist #1, etc.). For each of these roles for the task, e.g., role 1315c, corresponding actions 1315d and types or categories of virtual element renderings 1315e may be specified. One will appreciate that the correspondences between the tasks, roles and actions 1315d and AR element renderings 1315e may be recorded in a variety of forms (e.g., hierarchically, as shown here, in a relational database). A task-role-action may indicate what actions in accordance with the surgical plan are expected for a given team member role during a specific task. Similarly, the task-role-AR element table may indicate what, and how, elements from regions 1305a, 1305b, 1305c, 1305d are to be rendered in accordance with the surgical plan for a given team member role during a specific task.

The computer system managing the virtual elements and the surgical plan may maintain an internal data representation of the entire surgical theater so as to coordinate the member-specific renderings in accordance with the tables of FIG. 13B and the rules of FIG. 13A. For example, FIG. 13C is a schematic block diagram illustrating various data structures in an example global representation data structure 1320 of the surgical theater, as may be implemented in some embodiments. For example, each data structure may form a software object class, database, script, etc. The interior model data structure 1320a may provide a central record for the one or more interior models generated during the procedure, including a record of all the virtual elements appearing therein. The exterior model data structures 1320b may be the collection of virtual elements outside of the interior model (e.g., range 520c). Theater composition data structures 1320c may include a repository of virtual elements corresponding to deployed equipment or equipment contemplated for deployment (e.g., such as virtual equipment 1105h) in the theater, as well as whether the corresponding real-world element presently appears in the theater. Team member data structure 1320d may include information on the respective team members, such as the roles they can assume throughout the procedure in accordance with the surgical plan, their present pose, dimensions, etc. Surgical plan data structure 1320e may include the surgical plan and may be updated when adjustments are made throughout the procedure. Surgical metadata 1320f may include the patient history (including., e.g., CT scans), references to similar historical surgeries, etc.

FIG. 13D is a flow diagram illustrating various operations in an example process 1350 for managing role-based augmented reality depictions within a surgical theater, as may be implemented in some embodiments. At block 1350a, the computer system may determine the initial surgical plan (e.g., as created before the surgical operation via the interface of FIG. 12). At block 1350b, the computer system may prepare the initial state of the global representation, e.g., populating the various structures or FIG. 13C with their initial values. At blocks 1350c and 1350d, the system may monitor the surgery, updating the global structure representation as the interior models are created, personnel move about the theater, robotic system data becomes available, etc. At block 1350e, the system may determine the current task (e.g., based upon an automated recognition of the current surgical state from sensor data, via user input, via monitoring of the surgical plan state, etc.).

At blocks 1350f and 1350g, the system may then iterate over the various devices in the theater with augmented reality renderings (e.g., the surgeon console, member headsets, member tablets, etc.). At block 1350h, the system may determine the member associated with the device's role. For example, roles may be pre-specified in the surgical plan or the system may assign roles based upon the provided data used to populate the team data structures 1320d.

Once the system identifies the member's role for the current task at block 1350h, at block 1350i, the system may determine the current action the member is to perform based, e.g., upon the current state of the surgical plan and the corresponding action for the member's role in the task-role-action table. Similarly, at block 1350j, the system may determine the virtual elements to be rendered for the member based upon the task-role-AR element table as indexed by the surgical plan state (e.g., rendering either the internal virtual elements of situations 1120, 1135 [e.g., as when the member is a peer surgeon advising the surgeon at the console 155] or the external elements of situations 1125, 1140 [e.g., when the member is a technician being instructed to move an arm into position] depending upon the member's currently assigned action and role). At block 1350k, the system may then direct the rendering on the corresponding device in accordance with the member or device's current pose and the determinations of blocks 1350i and 1350j.

Once all the devices have been considered at block 1350f, the system may wait at block 1350l an appropriate interval before considering whether to perform another update. In some embodiments, updates may be triggered upon changes in the theater state, rather than, or in combination with, a timed delay. Once the system determines that the surgery is complete at block 1350c, then the system may perform various post-surgical recording and analysis operations at block 1350m as described in greater detail herein.

### Surgical Operations Planning—Role Based Data Siloing and Optimization—Visual Selectivity

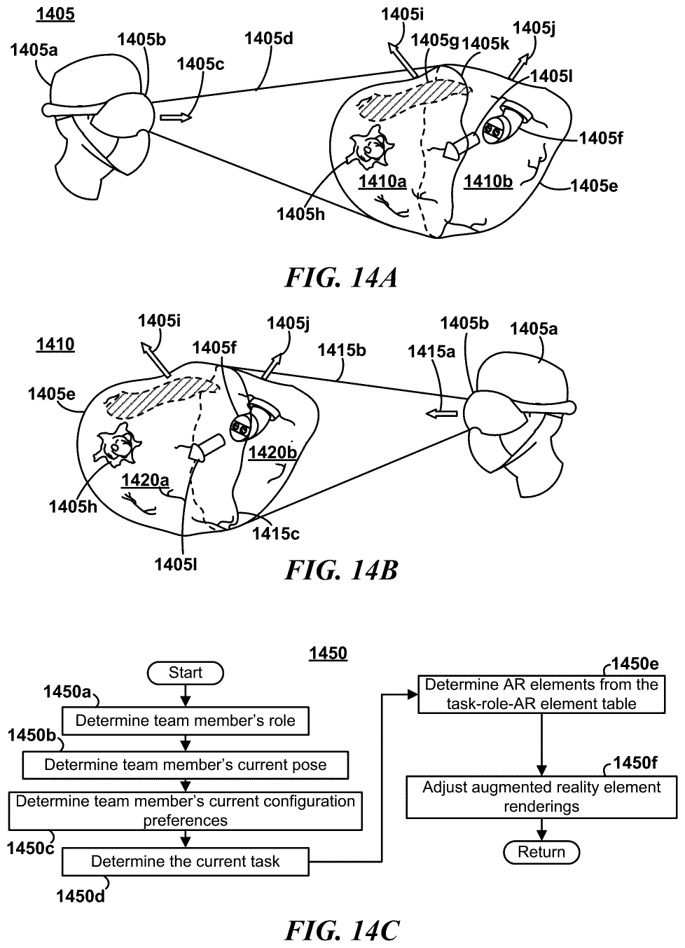

As discussed, various embodiments may adjust or limit various team members' augmented reality depictions in accordance with their current role in the surgical procedure. FIGS. 14A and 14B are schematic representations of a team member 1405a viewing an augmented reality representation of a patient interior model 1405e, using an augmented reality headset 1405b (or, mutatis mutandis, a tablet or other augmented reality device), from a first pose of situation 1405, and from a second pose of situation 1410 and consequent rendering adjustments, as may be implemented in some embodiments.

In the first pose of situation 1405, the member 1405a's field of view 1405d via the augmented reality device 1405b results in a division of elements in the interior model 1405e. Specifically, elements appearing in the volumetric region 1410a (e.g., the target anatomy 1405h, a portion of port placement region 1405g, tissue surfaces of the model, etc.) of the model 1405e may occlude elements in the volumetric region 1410b (e.g., the imaging device-bearing instrument virtual depiction 1405f or an associated imaging device viewing indication arrow 1405l, tissue surfaces in region 1410b, etc.). Thus, in some embodiments, in the perspective of the pose of situation 1405 the elements in region 1410a may not be rendered, or may be rendered at a reduced opacity, to facilitate the member's view of the elements in region 1410b as well, which may be rendered at full opacity. Conversely, in the pose of situation 1410, the rendering computer system may identify regions 1420a and 1420b, and the renderings correspondingly adjusted so that the member might view items in region 1420a at full opacity and those in region 1420b at a lesser opacity. In some embodiments, occluding items appearing in one of the “fully rendered” regions may still have their opacity adjusted, or an outline rendered upon the occluding item of the occluded item's boundaries, so that the member may still infer the state of relevant elements.

Such opacity adjustments or identifications of the regions 1410a, 1410b, 1420a, 1420b may occur in connection with rendering pipelines or by independent consideration of vectors associated with such pipelines. For example, the computer system may consider the vector 1405c associated with the augmented reality device's pose, relative to the normals of various items in the model to determine the opacity at which those items should be rendered. For example, the normal 1405i to the exterior of the model around region 1410a produces a negative dot product with the vector 1405c, whereas the normal 1405j to the exterior of the model around region 1410b produces a positive dot product with the vector 1405c. Comparison of such dot products may provide one method for readily identifying the volumes of regions 1410a and 1410b. Conversely, in the situation 1410, the new field of view 1415b vector 1415a may form a positive dot product with the vector 1405i and a negative dot product with the vector 1405j, resulting, generally, in a reversal of the opacity choices made for the pose of situation 1405. Normals at vertices or faces along the line 1405k (and line 1415c) may have dot products of zero or approximately zero relative to vector 1405c (and vector 1415a, respectively).

In some embodiments, however, where the user's current role, task, or action association implicates a particular virtual element or portion of a virtual element (e.g., as indicated in the task-role-AR element table), then the above-described rendering adjustments may be further adjusted so that the virtual element or portion of the element of interest to the member 1405a may be readily visible to the member 1405a. For example, in situation 1405 the entire model 1405e and all of its elements may have a default rendering of 80% opacity, the occluding region 1410a may be generally rendered at 30% opacity, but if the member is participating in port placement, the port placement recommendation region 1405g may be rendered at full opacity, or outlined, so that the member 1405a may readily infer the relative pose of the region 1405g.

For further clarity, FIG. 14C is a flow diagram illustrating various operations in an example role-aware rendering process 1450, as may be implemented in some embodiments. At block 1450a, the system may determine the team member's role (e.g., based upon pre-filled values in the team member data structure 1320d and the current state of the surgical plan). At block 1450b, the system may determine the member's current pose within the surgical theater (e.g., using the various registration methods disclosed herein). At block 1450c, the system may determine the team member's current preferences. For example, rather than call attention to virtual elements via opacity adjustments, some team members may prefer to identify virtual elements relevant to their current task via a billboard outline overlay.

At block 1450d, the system may determine the current surgical task, e.g., based upon the current state of the surgical plan. At block 1450e, the system may consult the task-role-AR element table to determine which AR virtual elements are associated with the current role. Some embodiments may also consider the particular action the member is currently performing via the task-role-action table and then refine the virtual element selection when the task-role-AR element table includes action-level specificity in the relevant list of AR element categories. At block 1450f, the system may adjust the augmented reality renderings based upon the member's pose and the virtual elements as determined at block 1450e.

### Augmented Reality Integration—Example Registrations—Local Device Data

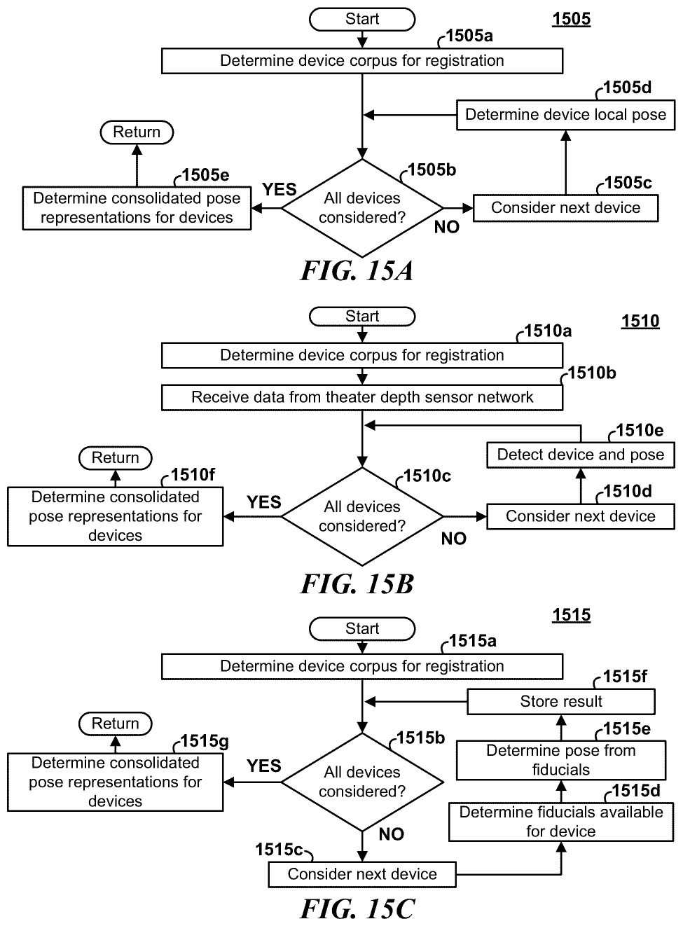

Registration of the various augmented reality devices within the theater may proceed in a variety of manners, various of which may be combined to achieve higher precision. For example, FIG. 15A is a flow diagram illustrating various operations in a first example augmented reality registration process 1505, as may be implemented in some embodiments. In this example, the augmented reality devices maintain their own local pose determinations relative to their local registration protocols (e.g., in accordance with the augmented reality device manufacturer's specification). The computer system (e.g., one of computer systems 190a and 190b) may avail itself of these local determinations to infer the global pose determination, e.g., relative to a robotic system as a common global reference in the theater 100b or to the surgical table in the theater 100a.

Specifically, at block 1505a, the system may determine the augmented reality devices in the theater for registration including, e.g., tablets, augmented reality headsets, mobile phones, etc. For clarity, the surgical instrument imaging device output may be registered, e.g., using the forward kinematics information provided by the instrument and robotic arm. The system may consider the devices to be registered at blocks 1505b and 1505c, collecting their local pose determinations at block 1505d. Once all the local determinations have been collected, at block 1505e the system may consolidate the local poses into the global coordinate reference frame of the theater. Some augmented reality devices may facilitate direct transformation operations between the respective coordinate reference frames (e.g., where the augmented reality device can transform its local pose to the global frame itself based upon gyroscope and accelerometer data following a common initialization). However, in some embodiments, where such direct transformation is not available, comparisons of depth data from the various augmented reality devices' respective fields of view may be used to infer relative poses (e.g., recognizing a common reference in the theater from the device's depth data using a particle filter or similar Monte Carlo method, machine learning system, etc.) and then the local poses transformed to the common global theater reference frame.

### Augmented Reality Integration—Example Registrations—Device Recognition

As another example, FIG. 15B is a flow diagram illustrating various operations in a second example augmented reality registration process 1510, as may be implemented in some embodiments. In this example, an in-sensor depth determination network is part of the surgical theater (e.g., a suite of sensors configured to capture depth data positioned around the surgical theater upon one or more of the ceiling, equipment surfaces, walls, etc.) and may be used to generate consolidated depth frames of the theater from the depth sensor device outputs. Such sensor suites may provide operating room awareness by, e.g., identifying object locations, personnel positions around the surgical table, additionally capturing visual images of the theater from various angles, etc. The consolidated depth representation from these sensors may then be aligned with the global reference frame of the surgical theater and inspected to locate particular augmented reality devices based upon their depth map signatures.

Again, at block 1510a, the system may determine the corpus of devices for registration. At block 1510b, the system may receive the current depth frame data of the surgical theater from the in-theater depth sensor network. At blocks 1510c and 1510d, the system may then iterate among the devices from block 1510a to be registered, seeking to detect the device in the sensor-network depth frame at block 1510e. For example, the surface “silhouette” or “signature” of the augmented reality device may provide a suitable target for a particle filter, machine learning system, etc., to determine a correspondence between a representation of the device in accordance with its product dimensions (e.g., as provided by the augmented reality device manufacturer) and with the corresponding portion of the sensor-network depth frame. A machine learning system (e.g., a neural network, support vector machine, random forest, ensemble collection of machine learning systems, etc.) may be used to recognize the identity and pose of the in-theater items at block 1510e (including the augmented reality devices) from depth data. For example, the machine learning system may be trained upon representations of the items' respective shapes and poses from virtual models, e.g., computer-aided design (CAD) models of known objects within the theater (e.g., robotic systems, robotic arms, instruments, surgical equipment, augmented reality devices, etc.). Perspective views of CAD models may be readily produced (e.g., applying appropriate scaling, rotation, and translation transforms within a rendering pipeline) to provide ground truth training and validation data sets for the machine learning system. In some embodiments, not only the depth representation of the items may be learned, but their textures and colors may also be recognized (e.g., using a You-Only-Look-Once (YOLO) network) to assist with registration.

Thus, such depth recognition may facilitate the recognition of augmented reality device poses directly from the depth data, as well as recognition of other objects and their poses in the theater (e.g., these other objects may likewise facilitate the AR device's registration, as when the system recognizes a portion of a robotic system in the depth data as a common reference). Where more than one augmented reality device of the same type appears within the theater, the different instances may, e.g., be distinguished by considering sensor data from the augmented reality devices (e.g., using the device's local depth data, or gyroscopic and accelerometer data, as discussed herein with respect to FIG. 15A), visual surface features of the device, radio fiducials, etc. In some embodiments, the type of robot or instrument used for the imaging device may not be known by the computer system. In these situations, some embodiments may also identify the robot type, the imaging device instrument, etc. based upon the visual image or depth-sensor data from the sensor network. Forward kinematics results for the imaging device instrument may then be inferred from the determined pose based upon the type of instrument and robotic system.

Once all the devices have been located (or at least those able to be located with the current depth data), the computer system may consolidate the devices' final respective pose determinations with the global coordinate frame of the theater, e.g., for later use by the computer system, at block 1510f (for clarity, where the sensor suite depth data was already aligned to the global coordinate system, then consolidation at block 1510f may not be necessary).

### Augmented Reality Integration—Example Registrations—Fiducial-Based

As yet another example registration process, FIG. 15C is a flow diagram illustrating various operations in a third augmented reality registration process 1515, as may be implemented in some embodiments, using fiducials located within the theater. The fiducials may include artificial visual markers, naturally occurring physical structures, radio markers, etc. prepared by a device manufacturer or placed within the theater at known locations by the team members. However, in some embodiments, the fiducials may also include real-time recognitions of various elements having known locations within the theater. For example, a hand gesture may be used to initialize registration, as when a team member assumes a specific pose in the global coordinate system, splays their fingers before them, and then invites all the augmented reality devices in the theater to register to that common reference (e.g., using an appropriately trained machine classifier, such as a YOLO neural network). Some embodiments may register poses of augmented-reality devices to the robotic system using, e.g., distinct features appearing upon the robot. In some embodiments, a fiducial may be placed upon the patient that is viewable by a sensor network or the robotic system (e.g., via an imaging device prior to insertion into the patient). Fiducials on the outside of instruments may also be used. Thus, in some embodiments the system may identify the robotic system's location and its forward kinematics for the imaging device and then invite a team member to place a fiducial marker upon the instrument itself or upon the patient's body to facilitate common registration.

In the depicted example, at block 1515a, the system may determine the augmented reality devices to be registered and iterate over this determined set of devices at blocks 1515b and 1515c. At block 1515d, the system may determine the fiducials available for the presently considered device, e.g., those which the device currently detects within its field of view or within its radio sensor range. Using these fiducials, a local pose determination may be inferred for the device relative to the fiducials at block 1515e and the result stored at block 1515f. Following consideration of all the devices, their poses relative to the fiducials may then be translated to the common global coordinate system, e.g., relative to a surgical robot (and hence facilitating registration of the imaging device's pose, e.g., based upon the robot's forward kinematics) at block 1515g. Again, such consolidation at block 1515g may already be effected if the fiducial poses in the global reference frame are already known and recognition likewise occurs in the global reference frame.

Again, while the above described processes 1505, 1510, and 1515 may be performed in isolation, in some embodiments they may be combined, or used in parallel, to infer more refined pose estimations during registration. For example, some systems may convert results from each of processes 1505, 1510, 1515 to a common global coordinate system and then perform particle filter and Bayesian prediction operations to align and consolidate the registrations for a more refined alignment. Similarly, actual fiducials appearing upon the equipment (e.g., quick response (QR) codes, registration patterns and graphics, textured surfaces, etc.) may also be used in combination with the recognition approaches (e.g., to validate or to refine the item recognition and pose determination of the other methods) to increase precision. Process 1510 and process 1515 may also be combined as part of an ensemble machine learning classification system, or independent machine learning systems may be trained for each of the processes and their results averaged (e.g., one classifier to recognize object depth signatures and poses from depth values as described above with respect to block 1510e, the other classifier to recognize object poses from recognized fiducials), etc. In some embodiments, these results may be then further verified via comparison with the results of process 1505 (e.g., via the particle filter and Bayesian prediction operations, as described above). Thus, one or more trained machine learning or localization systems may recognize the identities and poses of objects in the theater from, e.g., RGB image data, depth data, fiducial data, combinations thereof, etc.

### Augmented Reality Assisted Surgical Team Coordination—Virtual View

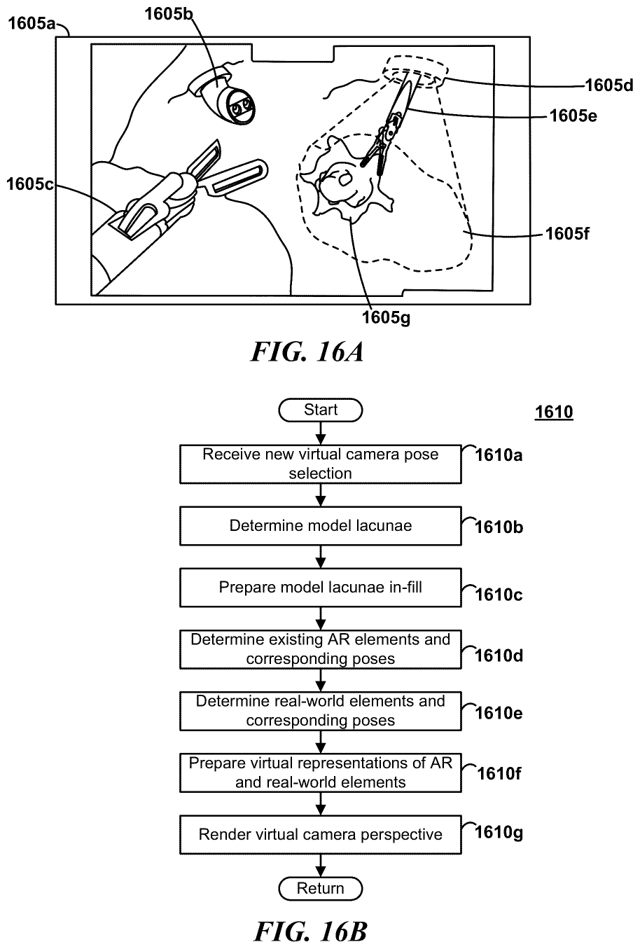

Some embodiments may further facilitate the planning of future port placements via a “virtual camera” or “virtual view” allowing, e.g., the operator 105c to assume the view of a not-yet-placed imaging device relative to a proposed port location or an arbitrary position within the interior model. For example, as shown schematically in FIG. 16A, a display, e.g., display 125, display 150, or display 160a, may render the view 1605a upon at team member's request, e.g., upon selection of a second proposed imaging device insertion location. In this view 1605a, the feed from the imaging device may be replaced (or presented in a separate window) with a rendering of the interior model from the perspective of the proposed “virtual imaging device” location.

Here, the operator 105c may perceive a representation 1605b of the currently deployed imaging device in its present pose. Proposed port locations, such as location 1605d, proposed instrument virtual rendering 1605e, and corresponding instrument motion range 1605f, previously rendered as augmented reality virtual elements over the imaging device feed, may now appear as virtual reality virtual elements within the interior model from the proposed virtual imaging device perspective. Similarly, the real-world target anatomy previously appearing in the imaging device view may now appear as a virtual element object 1605g, which may be a portion of the interior model sidewall. Virtual reality renderings of instrument virtual elements, such as the clasp 1605c, and additional port placements, may also be considered from this virtual position.

FIG. 16B is a flow diagram illustrating various operations in an example process 1610 for managing a virtual imaging device rendering, as may be implemented in some embodiments. For example, when requesting to transition from a current view of the imaging device output to a virtual rendering from a new perspective (or to render them in parallel), at block 1610a the system may receive the user's new virtual imaging device pose selection. This selection may be associated with a proposed port placement or may be an arbitrary position, e.g., which the operator is considering to access via an additional port. As discussed herein with respect to FIG. 3G, in some embodiments, at blocks 1610b and 1610c, the system may attempt to in-fill missing portions of the interior model to provide a more complete rendering for the virtual imaging device field of view.

At block 1610d the system may determine the existing poses of the virtual elements (e.g., port placement recommendation depictions 630b, 630c, the virtual port placement and augmented reality conical instrument range depiction icons 630d and 630e, etc.). Similarly, the system may determine real-world elements at block 1610e, such as surgical instruments (from whose forward kinematics a corresponding pose for rendering a virtual element, like clasp 1605c, may be inferred), target anatomy not self-evident from the interior model surface, etc. For the items and poses determined at blocks 1610d and 1610e, corresponding virtual element depictions may be determined at block 1610f. For example, a three-dimensional virtual element model of a real-world instrument (or corresponding SLAM results capturing the instrument), e.g., the rendering of the clasp 1605c, may be used for the real world instrument in the virtual rendering. Similarly, a composite virtual rendering of the target anatomy may be produced by merging the model interior surface with previously acquired data, such as a CT scan, to produce a more complete virtual depiction. Many augmented reality elements may already be in a form suitable for virtual rendering, as they are themselves three-dimensional models suitable for a graphics pipeline. However, as some augmented reality virtual elements may be rendered as overlays upon the real-world imaging device field of view (e.g., via billboards), corresponding three-dimensional models within the virtual view may need to be separately prepared at block 1610f. With the corresponding virtual elements ready for rendering, the elements may be appropriately translated, rotated, and scaled relative to the virtual view, then rendered relative to the model interior at block 1610g as part of the virtual imaging device perspective.

### Computer-Assisted Theater Planning

As mentioned, in some embodiments, the entire surgery may be pre-planned in virtual reality with a virtual patient and a virtual surgical theater, e.g., using elements in interface 1200 (though, as mentioned, planning, and updates to plans, via augmented reality may also be possible). During the surgery, the resulting surgical plan may then guide the in-theater visualization in a shared view. As discussed, this may be done entirely by “role playing” with the virtual elements, e.g., via the interface 1200 or via creation and manipulation of augmented reality elements within the theater. However, in some embodiments, the computer system may assist the users with their planning, providing warnings and predicting adverse events, before and in some embodiments during, the surgery.

FIG. 17 is a flow diagram illustrating various operations in an example process 1700 for operation-wide computer-assisted theater planning (e.g., in lieu of an exclusively “virtual augmented reality doll house” approach), as may be implemented in some embodiments. Team members may run the process 1700 before the surgery (e.g., at block 205b) to predict adverse events, such as collisions, inadequate port placements, etc. In some embodiments, the process may be re-run during the procedure (e.g., at block 205f, possibly in combination with the team's “virtual doll house” discussion), e.g., as adjustments are made, new circumstances arise, etc. In this manner, the operating team's “predictive horizon” for their surgical actions may be greatly extended both before and during the surgical procedure.

At block 1705a, before the surgery has begun, the computer system may receive contextual surgical data regarding the patient. For example, CT scans identifying target anatomy, past surgery data, health statistics (such as the patient's dimensions, body mass index, etc.), etc., may be provided for the patient or similarly situated patients. At block 1705b, the system may receive the task list for the surgery. Prior to beginning the surgery, the task list may be a standard task list associated with a given surgery. However, during the surgery, or during planning, new task lists may be prepared as the surgical plan changes.

At block 1705c, the system may also receive historical data from past surgeries performing the tasks of block 1705b. In some embodiments, only tasks appearing in the same surgery may be used, but in some embodiments and situations, the same task, even if it appears in different types of surgeries, may be considered. Such historical data may also include “rules” representing the collective experience of one or more team members and technicians from past surgeries. For example, it may be understood that certain combinations of port placements and instruments are unsuitable for certain tasks or for certain types of surgery or theater compositions. Accordingly, the historical rules may give such combinations lower priority or cause them to excluded from the team or from the system's consideration.

At block 1705d, the system may receive the team members' individual selections, e.g., via the interface of FIG. 12. For example, virtual objects relevant to one or more tasks to be performed may be introduced into the virtual theater. With this initial data in hand, at block 1705e, the system may then determine the initial virtual theater state, such as the location of equipment, personnel, robotic configurations, etc. during the first task of the surgery. The data from block 1705b may specify an initial port placement at block 1705f (e.g., for an imaging device instrument to be used for acquiring the interior model). In some embodiments, the user may specify the initial port placement, or the initial placement may be included a part of the initially received data if the surgery has already begun.

At block 1705g, the system may then determine the initial theater surgical plan. In some embodiments, the plan at block 1705g may be specified by the user for verification that no adverse events will occur during the simulation. For example, resolution of adverse events, such as collisions between robotic arms, instruments (within, or external, to the patient), equipment, personnel, etc. can be very difficult for a human operator to anticipate (e.g., absent roleplaying), and so a team member may propose a plan at block 1705g for verification by the computer system in the following steps.

The computer system may use historical data and forward project operations within the virtual theater, e.g., multiple times (e.g., using Monte Carlo based variations) to infer a “most ideal” order and character of actions for each of the tasks. Based on the initial plan, the run configurations for the surgical simulation may be prepared at block 1705h and the virtual procedure begun at block 1705i in accordance with the plan determined at block 1705g. Though only one simulation is shown in this example, one will appreciate that multiple simulations may be run in parallel in some embodiments (e.g., for different proposed initial plans).

As the simulation is run via iterations through blocks 1705j and 1705k, the system may update the virtual patient interior at block 1705l and composite virtual theater elements at block 1705m, e.g., of portions of interface FIG. 12, respectively. However, where the process is being used predictively, e.g., to evaluate whether adverse events occur, the iterations through block 1705j may occur very quickly (e.g., and not be rendered for the user's inspection). Such updates may include updates both to element renderings (when they will be presented to the user), but also updates to the corresponding physics models for the elements. Thus, the virtual elements may have their attributes (pose, configuration, etc.) updated at block 1705n.

In some embodiments, the system may seek to predict adverse events at block 1705o, testing for collisions, breach of spacing or placement rules, etc. Where such adverse events occur, the system may halt the simulation and notify the reviewing team member. The team members may then adjust the plan manually to avoid the adverse event and then rerun or continue the simulation. However, in some embodiments the system may first seek to revise the port placements at block 1705p (e.g., applying the processes 1000 or 1010) and consequently adjust the surgical plan for the theater at block 1705q so as to avoid the adverse event. Attempting an automated resolution before involving a team member may allow the team member to make a more appropriate manual intervention, as the system may be able to notify the team member if they are proposing a change the system already verified would result in the same or another adverse event.

When the simulation is complete at block 1705j, at block 1705r the system may present the simulation results for the reviewer's approval (e.g., in the form of a surgical plan) and receive any final manual adjustments at block 1705s. If the user has made adjustments at block 1705s, or simply desires to consider another plan variation, at block 1705t, the system may consider additional simulation to verify the proposed changes. However, if the reviewer instead approves the proposed plan, then at block 1705u the plan may be published, e.g., prepared for use in a surgery yet to begin, or as an adjustment to an existing surgical plan (which may already be underway in the real-world theater).

### Computer-Assisted Theater Planning—Creation of the Surgical Space Using Historical Data and Insights

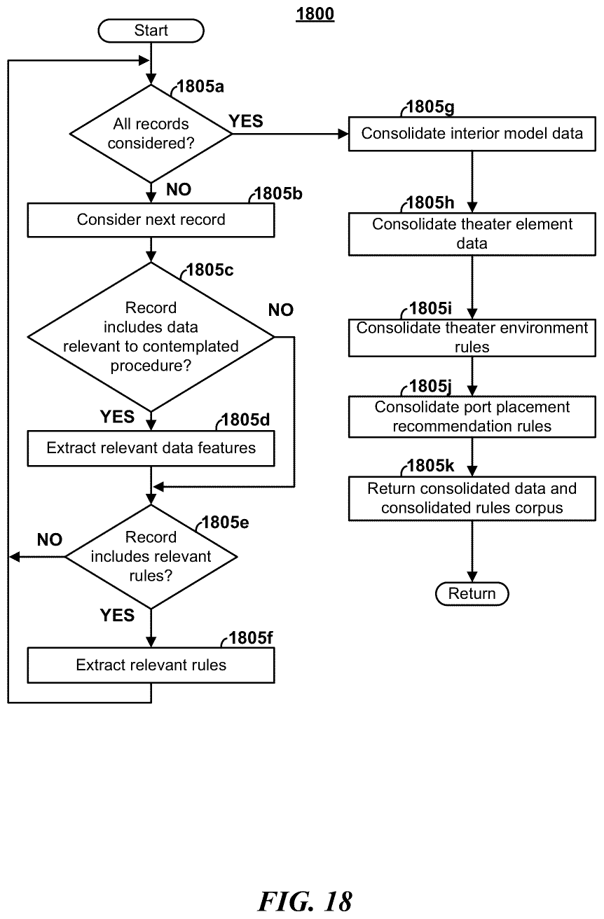

As discussed, various embodiments may take advantage of recorded procedural data and team member insights to inform the creation of a single world-aligned visualization of the surgical space, both inside and outside the patient body, which may be superimposed onto the real world. FIG. 18 is a flow diagram illustrating various operations in an example process 1800 for history-based virtual theater creation, as may be implemented in some embodiments (e.g., using a record of past surgeries' data as captured using software, firmware, or hardware, such as the Intuitive Surgical, Inc. dvLogger™). Historical data may include, e.g., the imaging device view, kinematic poses of patient cart and surgeon console, instrument positions and poses, system events and errors, interior model states, composite system states, surgical plan data, etc.

At blocks 1805a and 1805b, the computer system may iterate over the received historical records and determine at block 1805c if the record includes data relevant to the current procedure. For example, if the current surgery includes tasks appearing in the record, even if the tasks are occurring in a different surgery, the relevant data features from the record (e.g., theater states during adverse events, theater sates correlated with more effective or better outcomes, etc.) may be extracted at block 1805d.

At block 1805e, if the system determines that the record includes explicit rules relevant to the procedure (e.g., rules addressing specific actions in a task to be performed in the presently contemplated procedure), then at block 1805f the rules may likewise be recorded. After all the records have been considered, the system may perform a consolidation process so as to convert the acquired data into rules, constraints, objectives, etc. for consideration in planning the currently contemplated procedure. Where all the historical data is to be presented to the reviewer for consideration in their own planning, consolidation may include sorting the material (e.g., based upon surgical outcomes or other metrics discussed herein) and presenting the material in a readily searchable manner to the user. In this example, at block 1805g the system may consolidate any acquired interior model data, focusing on extrema (e.g., more port reinsertions may reflect a negative first choice and consequently the initial port placement in the model may precipitate a penalty for a corresponding location in the current procedure's recommendations). Similarly, at block 1805h, the system may consolidate composite theater element data, such as collisions (as may have been previously recognized at block 1705o) between robotic arms, equipment, personnel etc. or other adverse events. Corresponding conjunctions of elements in the current planning may then likewise be penalized to avoid similar adverse outcomes.

At block 1805i, the system may similarly consolidate the theater environment rules. For example, some rules may have been specifically appended to the record following a surgery so as to avoid the adverse events that occurred during the procedure, and associated with data in blocks 1805g or 1805h. Similarly, results for port placement may be consolidated at block 1805j. For example, the system may set as a rule a maximum distance for various instruments from a particular type of target anatomy (such as the one considered by the present surgery) or from an imaging device viewpoint.

At block 1805k, the system may then publish the consolidated data and rules corpus for use by the surgical planner (as well as, e.g., future detection of a breach at block 1705o) or for the user's consideration (e.g., as a warning when the user proposes a port placement or other action violating, or nearly violating, a rule).

### Data Gathering and Analysis

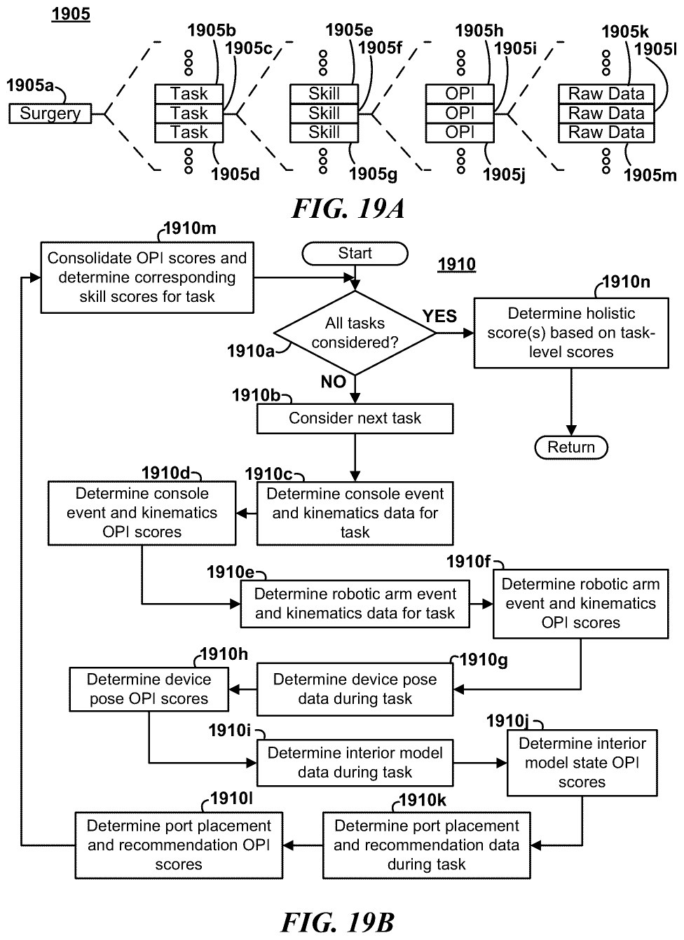

FIG. 19A is a schematic block diagram of various components in a surgical analysis framework 1905, as may be used in some embodiments. Such a framework may be a useful method for organizing surgical data, particularly so as to assess a surgeon's performance, or the performance of the entire surgical team. The data may include logged system and kinematics data from a robotics system, equipment and personnel poses throughout the surgery, instrument motions and states, patient vitals, etc. For example, the computer system may critique a proposed port placement by a team member using previous surgical scores for past surgeries employing similar placements. Similarly, port placements that produced better outcomes/efficiencies may be given priority when the system orders its recommendations.

Particularly, as discussed, data for an entire surgery 1905a may be broken into a plurality of tasks 1905b, 1905c, 1905d. Each task may, in turn, be broken down into an assessment of skills (e.g., suturing, cauterizing, closing, imaging device movement, etc.) employed in the action of the respective tasks (e.g., prostate removal, locate target anatomy, etc.). For example, while the overall quality of the surgery may depend upon the quality of each of the tasks, including tasks 1905b-d, the quality of each task, e.g., task 1905c, may depend upon the scores for the skills pertinent to that task (in this example, the task 1905c depending upon skills 1905e, 1905f, 1905g). Each skill may in turn be assessed based upon one or more functions of the raw data, such functions referred to as objective performance indicators (OPIs). Here, for example, the skill 1905f depends, a least in part, on the OPIs 1905h, 1905i, and 1905j. Similarly, the OPI function 1905i considers a plurality of raw data types including the raw data types 1905k, 1905l, 1905m. For example, the OPI “excessive camera movement” may consider the velocity, acceleration, and total motion of a camera over a fixed time interval. Some software, firmware, or hardware recording systems may also generate analytics, e.g., how long a procedure is, how much and what kind of personnel motion occurs, etc.

FIG. 19B is a flow diagram illustrating various operations in an example surgical assessment process 1910, as may be implemented in some embodiments (though this example focuses on a robotic surgical theater, one will appreciate that equivalent data, if available, may be used in a non-robotic theater). At blocks 1910a and 1910b, the system may iterate through all the tasks of a procedure. For each task, at block 1910c, the system may determine the console system event and console kinematics data, as well as the corresponding OPI scores at block 1910d. Similarly, at blocks 1910e and 1910f the system may determine robotic arm event and robotic kinematics data, as well as corresponding OPI scores. At block 1910g the computer system may determine augmented reality device pose data (e.g., user headsets, tablets, etc.) throughout the procedure and corresponding OPI scores at 1910h. At block 1910i the system may determine the interior model and corresponding OPI score at bock 1910j (e.g., an OPI assessing the area and number of holes in the interior model may be used as a proxy metric inversely indicating the quality of the surgeon's panning of the imaging device for interior model creation).

Finally, at block 1910k, port placements or recommendations for the same may be acquired and corresponding OPI scores generated at block 1910l. Example OPI scores generated at block 1910l may include: the amount of time from a port placement recommendation until creation of a port at the position (an OPI which may inform, e.g., a surgical efficiency skill); the amount of time during which the operator or team members viewed a proposed port placement before accepting the recommendation (an OPI which may inform, e.g., a surgical planning skill); etc. Such OPI scores, relative to the overall score for the task or surgery, may reveal correlations between port placement and surgical outcomes and efficiency. The computer system may analyze the interior model and port placements from past surgeries (e.g. 200 procedures) of a specific task or surgery type (e.g. a left colectomy) to recognize port placement patterns. A specific port placement pattern shape (e.g. linear distances between ports) may lead to differences in efficiency (e.g., procedure time) and surgical outcomes. Resemblance of a current placement to such patterns may itself provide an OPI value and port placements driving better outcomes and efficiencies may even be recommended as paradigmatic standards or recommendations for a surgery.

With the OPI scores available, the system may determine corresponding skill scores for the task at block 1910m. Once all the tasks have been considered, some embodiments may determine a score for the entire surgery at block 1910n (e.g., a sum of the task scores weighted by the relative importance or duration of the tasks in the surgical procedure).

### Data Gathering and Analysis—Visualization Record Keeping

The interior model, particularly where the model includes depictions of port placements or recommended port placements, may provide a useful and convenient visual representation for readily communicating and sharing port placement choices among patients, hospital staff, technicians, etc. Accordingly, various embodiments may include such visualizations for quick reference in surgical case reports, patient charts, surgical databases, etc.

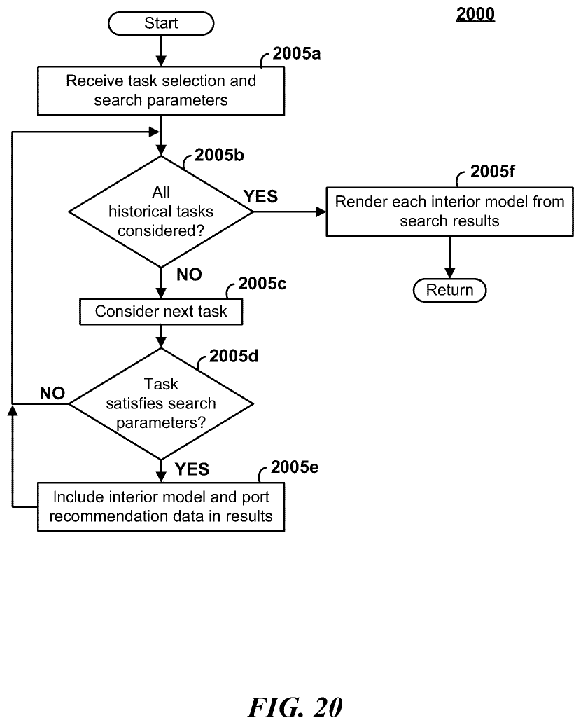

FIG. 20 is a flow diagram illustrating various operations in an example visual record search process 2000 contemplating such interior model based record keeping, as may be implemented in some embodiments. Specifically, after a plurality of surgeries, the interior models and corresponding port placements therein may be recorded for future reference. At block 2005a, the system may receive user search parameters across this corpus, indicating, e.g., the tasks performed while the interior model was being updated or port placement recommendations were generated or accepted. Search parameters may also include, e.g., the size of the interior model, restrictions on locations of the port placement recommendations, limitations on which of the recommendations was rejected or accepted, adjustments made by a team member to a proposed placement (e.g., as proposed via process 1000 or process 1010), the arm associated with the placement, the number of placements, etc.

At blocks 2005b, 2005c, and 2005d, the system may iterate over all the stored datasets, seeking those satisfying the requested task and other search parameters, temporarily storing interior models associated with such matching metadata at block 2005e (e.g., models and their recommended, or actual, port locations may be collected). After the available data has been considered, the models saved at block 2005e may be presented to the user for consideration at block 2005f. For example, a gallery grid of the interior models may be rendered (possibly including visualizations of their actual port locations or recommended locations), sorted in the order of models with the greatest number (or weighted sum) of metadata matches to the search terms of block 2005a. Such visualizations may be particularly useful as they may depict important aspects of the patient interior without sharing the patient's personal health information. Such anonymity may facilitate broader, anonymized data collection and review, which may be useful to, e.g., train machine learning systems, assess performance across many surgeries in a hospital network, etc. Such galleries may also be useful for peer-to-peer conversations as well as in conferences/conventions where case specific data is presented.

### Computer System

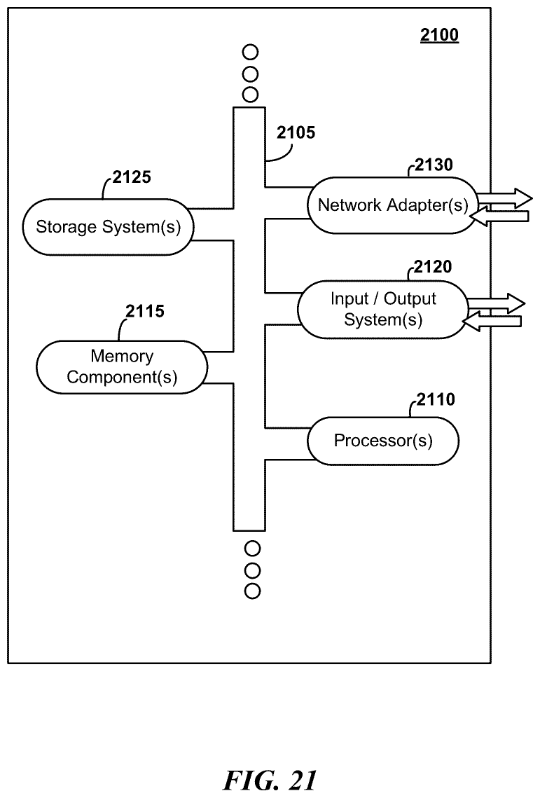

FIG. 21 is a block diagram of an example computer system as may be used in conjunction with some of the embodiments. The computing system 2100 may include an interconnect 2105, connecting several components, such as, e.g., one or more processors 2110, one or more memory components 2115, one or more input/output systems 2120, one or more storage systems 2125, one or more network adaptors 2130, etc. The interconnect 2105 may be, e.g., one or more bridges, traces, busses (e.g., an ISA, SCSI, PCI, I2C, Firewire bus, etc.), wires, adapters, or controllers.

The one or more processors 2110 may include, e.g., an Intel™ processor chip, a math coprocessor, a graphics processor, etc. The one or more memory components 2115 may include, e.g., a volatile memory (RAM, SRAM, DRAM, etc.), a non-volatile memory (EPROM, ROM, Flash memory, etc.), or similar devices. The one or more input/output devices 2120 may include, e.g., display devices, keyboards, pointing devices, touchscreen devices, etc. The one or more storage devices 2125 may include, e.g., cloud-based storages, removable Universal Serial Bus (USB) storage, disk drives, etc. In some systems memory components 2115 and storage devices 2125 may be the same components. Network adapters 2130 may include, e.g., wired network interfaces, wireless interfaces, Bluetooth™ adapters, line-of-sight interfaces, etc.

One will recognize that only some of the components, alternative components, or additional components than those depicted in FIG. 21 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 2130. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 2115 and one or more storage devices 2125 may be computer-readable storage media. In some embodiments, the one or more memory components 2115 or one or more storage devices 2125 may store instructions, which may perform or cause to be performed various of the operations discussed herein. In some embodiments, the instructions stored in memory 2115 can be implemented as software and/or firmware. These instructions may be used to perform operations on the one or more processors 2110 to carry out processes described herein. In some embodiments, such instructions may be provided to the one or more processors 2110 by downloading the instructions from another system, e.g., via network adapter 2130.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

We claim:

1. A computer-implemented method for presenting surgical port placement information, the method comprising:
receiving a three-dimensional representation of at least a portion of a patient interior;
determining a robotic arm's accessible surface relative to the three-dimensional representation; and
causing a potential port placement position to be rendered in association with the three-dimensional representation based upon the accessible surface.

2. The computer-implemented method of claim 1, the method further comprising:
causing a representation to be rendered indicating a range of motion of a surgical instrument relative to the potential port placement position, wherein the representation of the range of motion of the surgical instrument comprises a conical shape.

3. The computer-implemented method of claim 2, wherein,
dimensions of a first end of the conical shape correspond to the outer accessible limits of the surgical instrument, and wherein,
dimensions of a second end of the conical shape correspond to dimensions of a contemplated port aperture.

4. The computer-implemented method of claim 3, wherein,
at least a portion of the representation of the range of motion of the surgical instrument intersects the three-dimensional representation of the at least a portion of the patient interior, and wherein the method further comprises:
causing a representation of the intersection of the representation of the range of motion of the surgical instrument with the three-dimensional representation of the at least a portion of the patient interior to be rendered.

5. The computer-implemented method of claim 4, wherein,
causing a potential port placement position to be rendered comprises causing the potential port placement position to be rendered as an augmented reality element upon an imaging device output.

6. The computer-implemented method of claim 1, the method further comprising:
causing a representation of the accessible surface to be rendered upon the three-dimensional representation.

7. The computer-implemented method of claim 6, the method further comprising:
determining a plurality of potential port placement positions upon the accessible surface; and
determining a plurality of scores, at least in part, by determining a score for each of the plurality of potential port placement positions upon the accessible surface, and wherein,
the potential port placement position caused to be rendered in association with the three-dimensional representation is the potential port placement position of the plurality of potential port placement positions associated with a best score of the plurality of scores.

8. A non-transitory computer readable medium comprising instructions configured to cause at least one computer system to perform a method, the method comprising:
receiving a three-dimensional representation of at least a portion of a patient interior;
determining a robotic arm's accessible surface relative to the three-dimensional representation; and
causing a potential port placement position to be rendered in association with the three-dimensional representation based upon the accessible surface.

9. The non-transitory computer readable medium of claim 8, the method further comprising:
causing a representation to be rendered indicating a range of motion of a surgical instrument relative to the potential port placement position, wherein the representation of the range of motion of the surgical instrument comprises a conical shape.

10. The non-transitory computer readable medium of claim 9, wherein,
dimensions of a first end of the conical shape correspond to the outer accessible limits of the surgical instrument, and wherein,
dimensions of a second end of the conical shape correspond to dimensions of a contemplated port aperture.

11. The non-transitory computer readable medium of claim 10, wherein,
at least a portion of the representation of the range of motion of the surgical instrument intersects the three-dimensional representation of the at least a portion of the patient interior, and wherein the method further comprises:
causing a representation of the intersection of the representation of the range of motion of the surgical instrument with the three-dimensional representation of the at least a portion of the patient interior to be rendered.

12. The non-transitory computer readable medium of claim 11, wherein,
causing a potential port placement position to be rendered comprises causing the potential port placement position to be rendered as an augmented reality element upon an imaging device output.

13. The non-transitory computer readable medium of claim 8, the method further comprising:
causing a representation of the accessible surface to be rendered upon the three-dimensional representation.

14. The non-transitory computer readable medium of claim 13, the method further comprising:
determining a plurality of potential port placement positions upon the accessible surface; and
determining a plurality of scores, at least in part, by determining a score for each of the plurality of potential port placement positions upon the accessible surface, and wherein,
the potential port placement position caused to be rendered in association with the three-dimensional representation is the potential port placement position of the plurality of potential port placement positions associated with a best score of the plurality of scores.

15. A computer system comprising:
at least one processor; and
at least one memory comprising instructions configured to cause the computer system to perform a method, the method comprising:
receiving a three-dimensional representation of at least a portion of a patient interior;
determining a robotic arm's accessible surface relative to the three-dimensional representation; and
causing a potential port placement position to be rendered in association with the three-dimensional representation based upon the accessible surface.

16. The computer system of claim 15, the method further comprising:
causing a representation to be rendered indicating a range of motion of a surgical instrument relative to the potential port placement position, wherein the representation of the range of motion of the surgical instrument comprises a conical shape.

17. The computer system of claim 16, wherein,
dimensions of a first end of the conical shape correspond to the outer accessible limits of the surgical instrument, and wherein,
dimensions of a second end of the conical shape correspond to dimensions of a contemplated port aperture.

18. The computer system of claim 17, wherein,
at least a portion of the representation of the range of motion of the surgical instrument intersects the three-dimensional representation of the at least a portion of the patient interior, and wherein the method further comprises:
causing a representation of the intersection of the representation of the range of motion of the surgical instrument with the three-dimensional representation of the at least a portion of the patient interior to be rendered.

19. The computer system of claim 18, wherein,
causing a potential port placement position to be rendered comprises causing the potential port placement position to be rendered as an augmented reality element upon an imaging device output.

20. The computer system of claim 15, the method further comprising:
causing a representation of the accessible surface to be rendered upon the three-dimensional representation;
determining a plurality of potential port placement positions upon the accessible surface; and
determining a plurality of scores, at least in part, by determining a score for each of the plurality of potential port placement positions upon the accessible surface, and wherein,
the potential port placement position caused to be rendered in association with the three-dimensional representation is the potential port placement position of the plurality of potential port placement positions associated with a best score of the plurality of scores.
